(19) United States
(12) Patent Application Publication
(10) Pub. No.: US 2007/0271623 A1
(43) Pub. Date: Nov. 22, 2007

(54) Plant Disease Resistance and Sar Regulator Protein

(75) Inventors: Erik Andreasson, Sverige (SE); Peter Brodersen, Dyssegaard (DK); Tom Jenkins, Drager (DK); John Mundy, Valby (DK); Nikolaj H.T. Petersen, Kobenhavn (DK); Stephan P. Thorgrimsen, Kobenhavn (DK); Anne Rocher, Frederiksberg (DK)

Correspondence Address: MERCHANT & GOULD PC, P.O. BOX 2903, MINNEAPOLIS, MN, 55402-0903, US

(73) Assignee: UNIVERSITY OF COPENHAGEN, Copenhagen (DK)

(21) Appl. No.: 10/596,010

(22) PCT Filed: Nov. 26, 2004
(86) PCT No.: PCT/DK04/00822

§ 371 Date: Mar. 13, 2007

Related U.S. Application Data
(60) Provisional application No. 60/526,319, filed on Dec. 1, 2003.

(30) Foreign Application Priority Data
Nov. 28, 2003 (DK) PA200301759

(51) Int. Cl. A01H 5/00; C12Q 1/68; C12N 9/12; C12N 15/82

(52) U.S. Cl. 800/265; 435/320.1; 436/501; 530/388.5; 800/279; 800/288; 800/301; 435/194; 435/6

(57) ABSTRACT

The invention provides a transgenic plant having increased expression of a positive regulator protein of systemic acquired resistance (SAR), thereby enhancing the SAR response and pathogen resistance of the plant. The positive regulator protein is a component of a signal transduction pathway leading to (SAR), and is a MAP kinase protein (MPK4) substrate, and interacts with transcription factors.

### FIELD OF THE INVENTION

The invention relates to broad spectrum disease resistance in plants and the identification, isolation and use of a novel regulator protein of systemic acquired resistance (SAR).

### BACKGROUND OF THE INVENTION

Disease resistance is a primary determinant of crop yield, and monocultures of genetically uniform plants are particularly vulnerable to attack by pathogens to which they have low natural resistance. A key parameter in plant breeding is thus the selection of plants exhibiting broad range, as well as specific resistance to diseases caused by infectious agents, including viruses, bacteria and fungi. Pathogen attack can be perceived by a host plant through the specific recognition of pathogen-derived molecules. This in turn elicits a rapid, localised, hypersensitive response by the plant, in the form of rapid necrosis at the point of pathogen attack. The host-pathogen interaction also induces a plant immune response known as systemic acquired resistance (SAR), which provides long lasting protection against a spectrum of pathogens in the uninfected parts of the plant (Yang et al., 1997, Genes Develop., 11: 1621-1639). Induction of SAR is thought to rely on the release of one or more signal molecules, including salicylic acid (SA), at the site of infection and their movement throughout the plant via the phloem. Perception of this systemic signal by target cells leads to the coordinate expression of a subset of pathogenesis-related (PR) genes, which contribute to building and maintaining disease resistance. Exogenous application of SA appears to be sufficient to induce SAR and PR gene expression, while depletion of SA, by in planta expression of bacterial salicylate hydroxylase (NahG), suppresses SAR (Gaffney et al., 1993, Science 261: 754-756). Genetic screens, conducted in Arabidopsis to select mutants in the signal transduction pathway leading to SAR, have provided a fruitful approach to identify potential positive and negative regulators of SAR. Some mutants show enhanced disease susceptibility, either due to a failure to accumulate SA, for example eds1 (Falk et al., 1999, Proc Natl Acad Sci USA, 96: 3292-3297), or a failure to perceive SA and induce PR gene expression, as exemplified by the npr1 mutant (Cao et al., 1997, Plant Cell, 88: 57-63). The npr1 mutants (also known as the nim1 non-inducible immunity mutant), carry mutations in a gene encoding NPR1 protein, which comprises ankyrin repeats that facilitate protein-protein interactions. NPR1 is believed to interact with basic leucine zipper transcription factors that bind and regulate expression from PR gene promoters (Zhang et al., 1999, Proc Natl Acad Sci USA, 96: 6523-6528).

Other mutants, identified by genetic screening, display enhanced disease resistance. Lesion mimic mutants which constitutively express SAR and develop spontaneous necrotic lesions in the absence of pathogen challenge are common; however these may result from pleiotropic disruption of cellular homeostasis (Molina et al., 1999, Plant J. 17: 667-678). Constitutive defence mutants (cpr) have also been found which show elevated SA levels and constitutive PR gene expression, without forming spontaneous necrotic lesions (Bowling et al., 1994, Plant Cell 6: 1845-1857; Clarke et al., 1998, Plant Cell 10: 557-569). PR gene expression in these cpr mutants is dependent on the SA signal.

Mutant screens have identified two negative regulator genes of SAR, namely SNI1 and MPK4. sni1 mutations, which cause enhanced SAR, are likely to regulate SA perception, since the sni1 (suppressor of no-immunity) mutation can restore SAR in npr1 mutants, which are otherwise unable to respond to SA application by inducing SAR (Dong et al., 2001, Novartis Foundation Symposium 236:165-173). The Arabidopsis MPK4 gene encodes a Mitogen-activated Protein kinase 4 (MPK4) that under non-pathogenic conditions, constitutively represses SAR. Mutations in the MPK4 gene lead to increased SAR, as measured by enhanced SA levels and PR gene expression, and greater resistance to both bacterial and oomycete pathogens (Petersen et al., 2000, Cell 103: 1111-1120). The expression of at least 16 genes, including 8 PR genes, is significantly increased in mpk4 mutants, consistent with a constitutive SAR phenotype, while expression of certain jasmonic acid (JA)-induced genes is blocked. The constitutive SAR of mkp4 mutants is dependent on SA, and is abolished by in planta expression of bacterial salicylate hydroxylase. The mkp4 Arabidopsis mutant is characterised by a dwarf habit, but the plants do not form spontaneous lesions. Mutants homozygous for both mpk4 and npr1-1 are dwarf and constitutively express PR genes and SAR as in mpk4 mutants, while showing the SA hypersensitivity typical of npr-1, suggesting that MPK4 and NPR1 may be components of independent disease resistance pathways. Unlike NPR1, MPK4 appears to be involved in cross-talk between the JA- and SA-induced gene expression. While both MPK4 and NPR1 proteins regulate plant disease responses, they are believed to control the coordinate expression of different subsets of PR genes. Those PR genes regulated by MPK4 have been found to share similar cis-elements in their promoter sequences that may regulate their coordinate expression, but which are distinct from NPR1 regulated PR genes (Petersen et al., 2000, supra). One of these elements, called a W-box, is a consensus binding-site for plant-specific WRKY transcription factors (Eulgem et al. 2000 TIPS 5: 199-206) that has been shown to act as a silencing element in the promoter of the PR1 gene (Lebel et al. 1998 Plant J. 16: 223-33)

Several approaches are proposed to enhance the broad-spectrum disease resistance of crop plants. WO 9749822 describes the isolation of the NIM1 gene, and its expression in transgenic plants in order to increase PR gene expression and thereby enhance SAR. WO 01/66755 and WO200053762 describe the isolation of various plant homologues of the Arabidopsis NIM1 gene and their expression in transgenic plants to enhance SAR. Similarly, WO2000028036 describes transgenic plants expressing the NPR1 gene conferring enhanced SAR. An alternative approach to increase SAR in plants is described in WO2001002574 and involves silencing expression of the gene encoding the SNI1 negative regulator polypeptide. Silencing or blocking the activity of MPK4, a second negative regulator of SAR, in order to enhance broad resistance to plant pathogens is disclosed in WO 01/41556.

It is generally recognised that wide spread use of pesticides is a standard agricultural practise which is to the detriment of the environment, and the accumulation of their residues in ground water is a serious man-made problem. Hence there is a strong desire throughout the world to reduce agricultural dependence on chemical pesticides, and to focus on enhancing the inherent resistance of plants to disease by breeding and genetic engineering. The production of crop plants with improved broad range resistance to plant pathogens relies on the identification of plant genes and their respective proteins products, whose expression determines the level and extent of immunity to pathogen attack. In particular plant genes which are components of one of more disease resistance signalling pathway, i.e., are involved in their regulation, can provide useful tools to control the timing or level of a given defence response. The value of this approach is clearly exemplified by the examples given above, where modulated expression of SAR regulatory genes in transgenic plants can enhance resistance to various pathogens. It is preferable to modulate the expression of a positive regulator of SAR, since techniques designed up-regulate gene expression in a transgenic plant are generally more effective than those required to achieve complete silencing of gene expression. It is particularly desirable that any improvement in pathogen resistance attained in the transgenic plant is not accompanied by the formation of lesions due to a spontaneous hypersensitive response, since this will be highly disadvantageous to both the yield and quality of the crop. It is furthermore desirable to identify genes, which can be used to increase plant resistance to a wide range of natural pathogens, without impairing the plants ability to respond to and survive other predators or environmental stresses.

### SUMMARY OF THE INVENTION

The present invention is based on the identification of a positive regulator protein of systemic acquired resistance (SAR) in plants. MKS1 is shown to be an integral component of the SAR signal transduction pathway, interacting with other components of the pathway and positively regulating SA synthesis and PR gene expression. Enhancing the expression of this plant regulator protein is shown to increase SAR in plants and to increase their resistance to pathogen attack.

Accordingly, the invention provides a transgenic plant having enhanced disease resistance and increased expression of a positive regulator of systemic acquired resistance (SAR), characterised by a transgene encoding a MAP kinase substrate 1 (MKS1) polypeptide having an amino acid sequence comprising:
a. MAP kinase interaction domain 1 with sequence:
   IXGPRPXPLXVXXDSHXIKK and
b. transcription factor interaction domain 2 with sequence:
   PVVIYXXSPKWHXXXXEFMXWQRLTG, or
conservatively modified variants of said domain 1 and/or domain 2 sequence, wherein X refers to any amino acid residue.

In one embodiment the transgenic plant of the invention is characterised by a transgene having a nucleic acid sequence encoding a MKS1 polypeptide comprising an amino acid sequence selected from the group: SEQ ID No. 2, 6, 10, 14, 16, 20, 26, 27, 28 and conservative variants thereof.

In a further embodiment the transgenic plant of the invention, is characterised by a transgene encoding a MKS1 polypeptide, said transgene comprising a nucleic acid molecule having a nucleic acid sequence selected from the group: SEQ ID No. 1, 5, 9, 13, 15, and 19.

Another embodiment of the invention is directed to the use of a nucleic acid molecule that hybridises at high stringency to a nucleic acid molecule having a nucleic acid sequence selected from the group consisting of: SEQ ID No. 1, 5, 9, 13, 15, and 19, as a transgene to produce the transgenic plant of the invention having enhanced disease resistance and increased expression of a positive regulator of systemic acquired resistance.

Furthermore the transgene of the transgenic plant of the invention may comprise a homologous promoter, or alternatively the transgene may be a chimeric gene comprising a heterologous promoter selected from the group: constitutive promoter, tissue specific promoter, and inducible promoter.

The transgenic plant of the invention includes either a dicotyledonous or a monocotyledonous plant and seed from the transgenic plant.

In a further aspect of the invention is provided a method for producing the transgenic plant of the invention, characterised by introducing an expression cassette, comprising the transgene encoding the MKS1 polypeptide, into a plant and selecting the transgenic plant and its progeny expressing said MKS1 polypeptide. Furthermore the invention encompasses a recombinant vector comprising said expression cassette and the introduction of said expression cassette into a plant through transformation or via a sexual cross with a transformed plant.

In another embodiment the invention provides a method for detecting increased expression of MKS1 polypeptide in the transgenic plant of the invention, characterised in reacting an anti-MKS1 antibody with a protein extract derived from said plant. Furthermore the invention encompasses both a polyclonal and a monoclonal anti-MKS1 antibody.

In another embodiment the transgenic plant of the invention may be used for the cultivation of a crop, wherein said crop encompasses plant biomass generated by the growth of a seed or seedling, and includes reproductive parts, e.g. seed, caryopsis, cob, or fruit and vegetative parts, e.g. leaf and tuber.

In a further embodiment the transgenic plant of the invention is used in a breeding program, wherein a plant selected in the breeding program comprises the transgene having a nucleic acid sequence encoding a MKS1 polypeptide.

### BRIEF DESCRIPTION OF THE FIGURES

FIG. 1. Arabidopsis MPK4 and MKS1 interacting proteins.

A. Yeast two hybrid screening of an Arabidopsis cDNA Library with MPK4 as bait (BD fusion) identified MKS1 as an interacting prey (AD fusion), and screening with MKS1 as bait identified WRKY 25 and 33 as interacting prey (AD fusion). A directed two-hybrid assay (given in italics) between MKS1 as bait and MPK4 as prey, confirmed their interaction. Two-hybrid assays (in italics) between MKS1 as bait (BD fusion) and MPK3, 5, 6 and 17 as prey (AD fusion), as well as MKS1 or MPK4 as bait (BD fusion) and WRKY26, WRKY29 or WRKY25, WRKY33 as prey (AD fusion), respectively, showed no interactions. Yeast cells in the two hybrid screen were selected on the indicated nutrient depleted growth-media (-Histidine; -Leucine; -Adenine-Tryptophan) and assayed for β-galactosidase (β-gal) reporter gene activity.

B. ClustalW alignment of the amino acid sequence of Arabidopsis MKS1 (Acc.No:At3g18690) and homologues or orthologues from Brassica oleracea (Acc.No:BoBH544707 and BoBOHBT92TR+BOGQI24TF), Glycine max (Acc.No:GmBE020960), Arabidopsis (Acc.No:At1 g21326; At1g68450, At2g41180, AtAL138658, At2g44340, AtT46022, At2g42140, AtAL390921) and Oryza sativa (Acc.No:OsCAD40925; OsBAC15955; OsAP004654, Os8360.t05160, Os8355.t00567, OsAP003260), Nicotiana tabacum (Ntacrel 69), Zea mays (Acc.No:ZmBM340911, ZmCC442903, ZmCC613160, ZmCC635639, ZmCC661221, ZmCC700850), Medicago truncatula (Acc.No:MtAC143340.1). Identified and putative phosphorylation sites (SP) in MKS1 are indicated in italics. C-termini of the three MKS1 truncations and the Pep22 sequence are indicated above the MKS1 sequence. Aligned identical or equivalent amino acid residues are box-shaded. The consensus sequence of MKS1 is given below the alignment in bold, wherein Domain1 and 2 are underlined.

FIG. 2. In vitro interaction and phosphorylation of MKS1 by MPK4.

A. 35S methionine-labelled MPK4 (lane 1), and its binding to MKS1-GST fusion protein (lane 3), but not to GST protein alone (lane 2), following separation by SDS-PAGE and detection by phosphoimager.

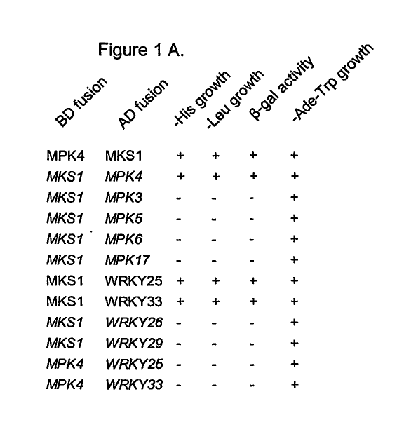

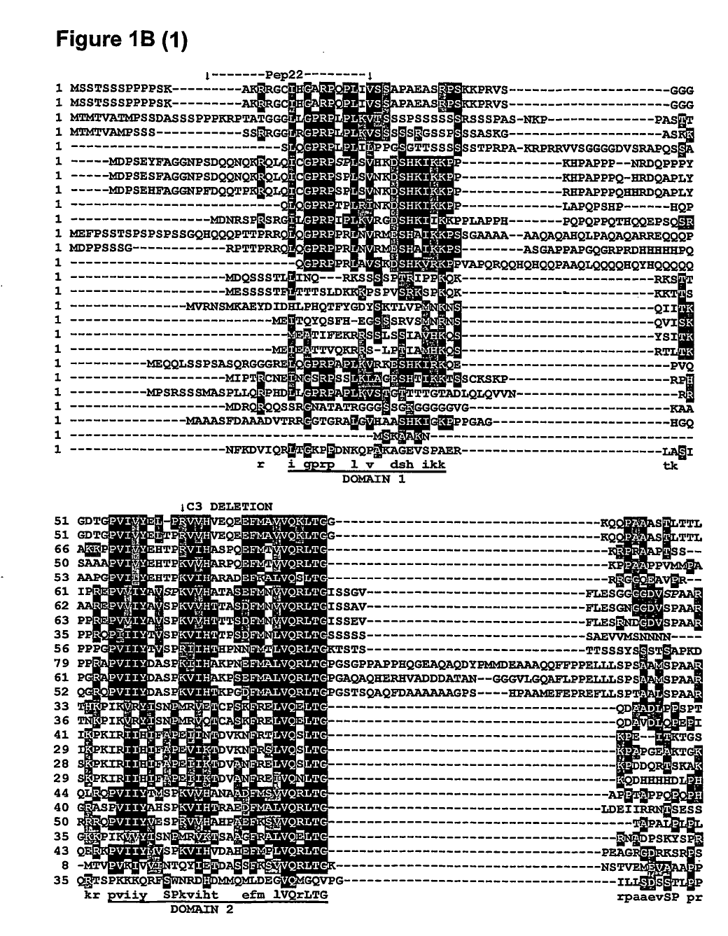

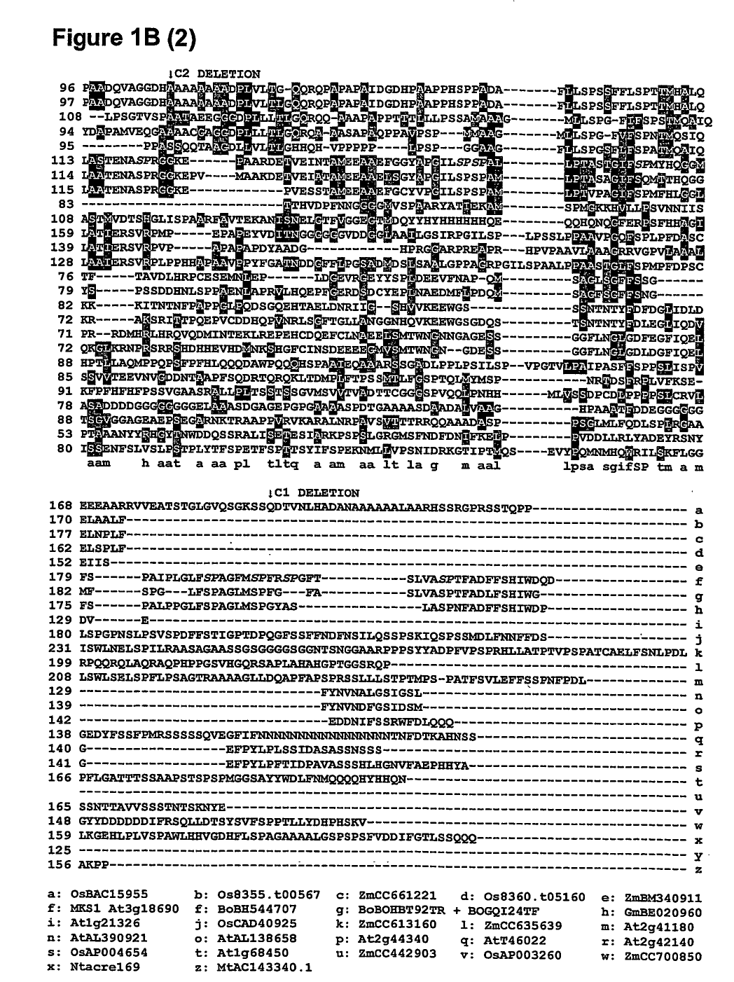

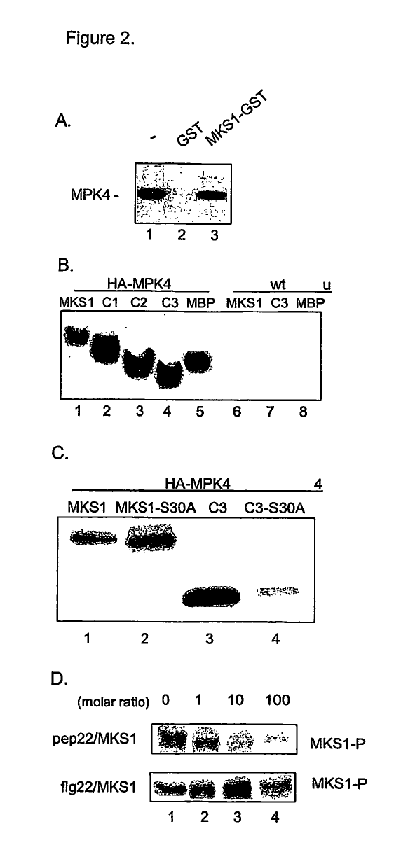

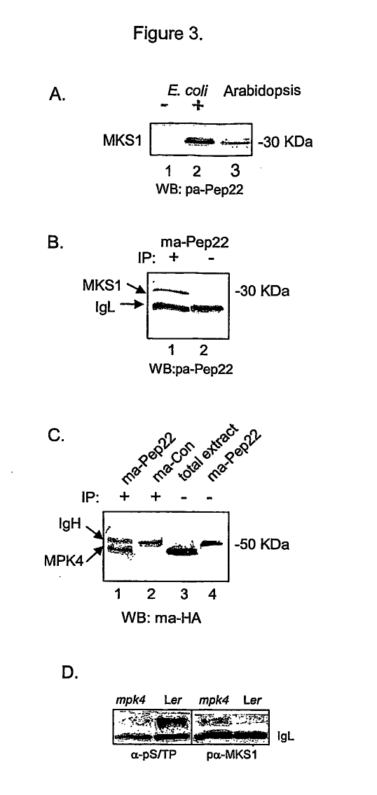

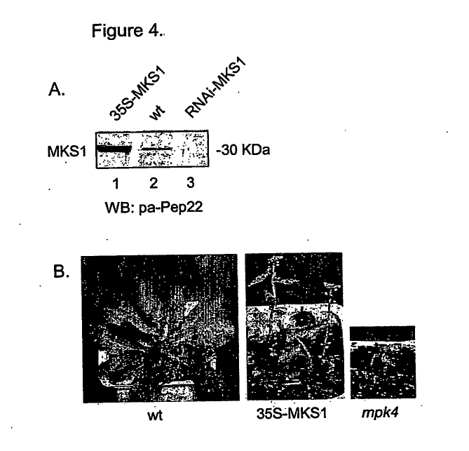

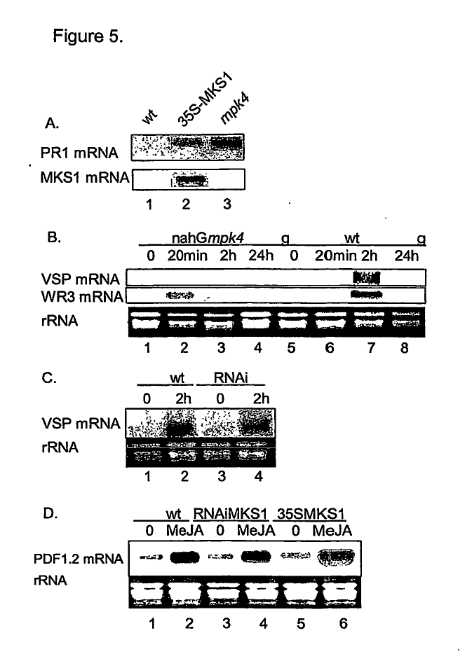

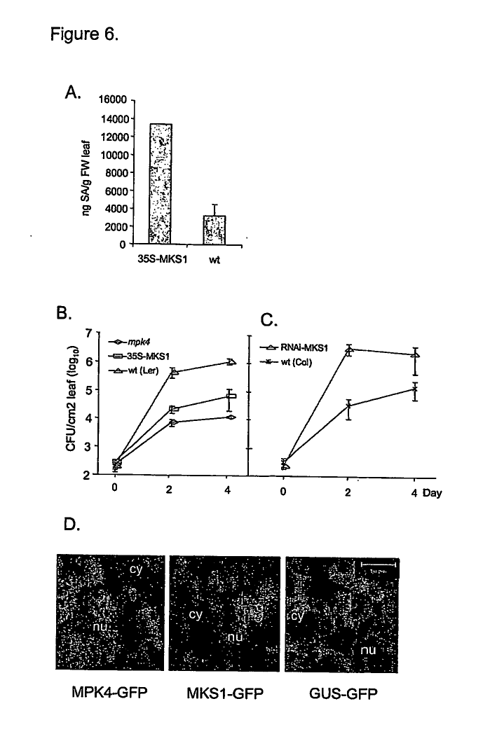

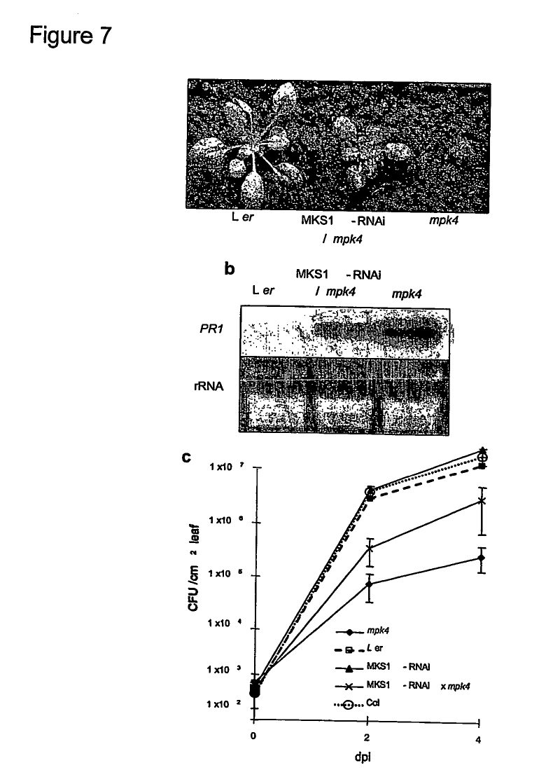

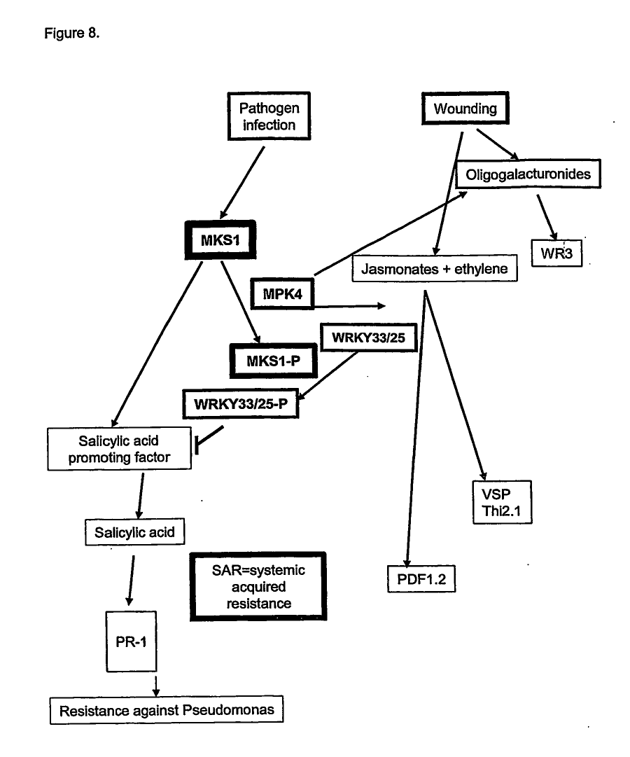

B. Phosphorylation assay with recombinant, full-length MKS1 (lane 1), C-terminal MKS1 truncations C1-C3, identified in FIG. 1B (lanes 24), or positive control myelin basic protein (MBP, lane 5) and HA-tagged MPK4 immunoprecipitated from transgenic plants, analysed by SDS-PAGE and phosphoimager detection. Control phosphorylation assays were performed with HA-antibody immunoprecipitates of non-transgenic, wild-type (wt) plants (lanes 6-8).

C. Phosphorylation assay with recombinant, full-length MKS1 (lane 1); mutant full-length MKS1-S30A (lane 2), MKS1 C3-truncation (lane 3), or mutant MKS1-S30A C3-truncation (lane 4) and HA-tagged MPK4 immunoprecipitated from transgenic plants, and analysed as in (B).

D. Top: Phosphorylation assay with recombinant, full-length MKS1 alone (lane 1) or in the presence of increasing molar ratios of Pep22, indicated in FIG. 1B (lanes 2-4) by HA-tagged MPK4, immunoprecipitated from transgenic plants, and analysed as in (B). Bottom: the phosphorylation assay (D. Top) was repeated with increasing molar ratios of a 22 amino acid peptide FLG22, as a negative control.

FIG. 3. In planta interaction of Arabidopsis MKS1 and MPK4.

A. Immuno-detection of MKS1 in extracts of E. coli before (lane 1) and after (lane 2) IPTG induction, and in an extract of wild type Arabidopsis rosette leaves (lane 3) by polyclonal anti-Pep22 antibody in a Western blot (WB: pa-Pep22).

B. Immuno-detection of MKS1 immunoprecipitated (IP) with monoclonal anti-Pep22 (ma-Pep22) from wild type plant extract (lane 1) or control sample lacking plant extract (lane 2) by polyclonal antibody pa-Pep22 in a Western blot (WB: pa-Pep22).

C. Immuno-detection of HA-MPK4 by anti-HA antibody (Western blot; WB: ma-HA) in immunoprecipitates (IP) of Arabidopsis plant extracts using anti-Pep22 antibody, ma-Pep22 (lane 1); negative control monoclonal antibody, ma-Con (lane 2); or in a total protein plant extracts (lane 3), and a mock extract, comprising buffer and maPep22 antibody (lane 4).

D. Immuno-detection of MKS1 in a Western blot with either phosphoserine/phosphothreonine antibody (α-pS/TP), or polyclonal antibody (pα-MKS1), following MKS1 immunoprecipitation from extracts of rosette leaves of wild type Arabidopsis (Ler) or mpk4 (mpk4) plants using monoclonal anti-Pep22 against MKS1.

FIG. 4. Transgenic Arabidopsis plants with modified MKS1 expression

A. Immuno-detection of MKS1 with polyclonal antibody pa-Pep22 (Western blot; WB: pa-Pep22) in extracts of 35S-MKS1 transgenic Arabidopsis (lane 1), wild type Arabidopsis Ecotype Col (wt; lane 2) and RNAi-MKS1 transgenic Arabidopsis (lane 3) plants.

B. Growth phenotype of wild type Arabidopsis Ecotype Ler (wt), 35S-MKS1 transgenic Arabidopsis and mpk4 mutant Arabidopsis plants.

FIG. 5. Effect of MKS1 and MPK4 on expression of defense and wounding response genes in Arabidopsis

A. RNA blot detection of PR1 and MKS1 mRNA in Arabidopsis wild type Ecotype Ler (wt; lane 1), 35S-MKS1 transgenic (lane 2) and mpk4 mutant (lane 3) plants.

B. RNA blot detection of VSP and WR3 mRNA accumulation in rosette leaves from nahGmpk4 (lanes 1-4) and wild type Ecotype Ler plants (wt; lanes 5-8), at different times after wounding.

C. RNA blot detection of VSP mRNA in rosette leaves from wild type Ecotype Col (lanes 1 and 2) and RNAi-MKS1 plants (lanes 3 and 4), at 0 h and 2 h after wounding.

D. RNA blot detection of PDF1.2 mRNA in wild type Arabidopsis Ecotype Ler (wt; lane 1 and 2), RNAi-MKS1 (lane 3 and 4), and 35S-MKS1 transgenic (lane 5 and 6) plants, at 0 h and 48 hr after methyl jasmonate (MeJA) treatment.

FIG. 6. Properties of Arabidopsis plants with altered MKS1 expression

A. Salicylate levels (ng/g FT (fresh weight)) in leaves from 4-week-old 35S-MKS1 transgenic Arabidopsis and wild type (wt) plants grown in soil. Error bars show standard deviation of triplicates; absence indicates insignificant differences.

B. Pathogen virulence assay of 4-week-old wild-type Arabidopsis Ecotype Ler (wt), 35S-MKS1 transgenic Arabidopsis and mpk4 mutant Arabidopsis plants inoculated with the virulent strain DC3000 of Pseudomonas syringae pv. tomato at a concentration of 1×10⁵ colony-forming units per ml (CFU/ml). Values represent average and standard deviations of cfu extracted from leaf disks in three independent samplings.

C. Pathogen virulence assay of wild type Arabidopsis Ecotype Col (wt) and RNAi-MKS1 transgenic Arabidopsis plants. Values given. are as in B.

D. GFP fluorescence detection of the GFP fusion proteins: MKS1-GFP, MPK4-GFP and GUS-GFP expressed in leaf mesophyll cells of transgenic Arabidopsis plants using confocal microscopy. Subcellular compartments indicated are: cytoplasm (cy); nucleus (nu).

FIG. 7. Suppression of mpk4 by MKS1-RNAi. a. Phenotypes of wild type (Ler), mpk4 carrying MKS1-RNAi (mpk4/MKS1-RNAi), and mpk4. b. RNA blot detection of PR1 mRNA in wild type (Ler), mpk4/MKS1-RNAi, and mpk4. c. Pathogen virulence assay of 4 week-old Arabidopsis wild type Ecotypes Ler and Col, mpk4, MKS1-RNAi, and mpk4/MKS1-RNAi inoculated with the virulent strain DC3000 of Pseudomonas syringae pv. tomato at a concentration of 1×10⁵ colony-forming units per ml (CFU/ml). Values represent average and standard deviations of cfu extracted from leaf disks in three independent samplings.

FIG. 8. A model of defense signaling in Arabidopsis, highlighting MPK4, MKS1, WRKY25 and WRKY33.

### DETAILED DESCRIPTION OF THE INVENTION

I. Abbreviations

GST: Glutathione-S-transferase

MKS1: Map Kinase Substrate 1

MPK4: Mitogen-Activated Protein Kinase 4

NahG: bacterial salicylate hydroxylase

PR gene/protein: Pathogen Related gene/protein

SA: Salicylic Acid

SAR: Systemic Acquired Resistance

WB: Western Blot

WT: wild type

II. Definitions

Agrobacterium-mediated transformation: is a technique used to obtain transformed plants by infection with Agrobacterium tumefaciens. During the transformation process the bacteria transfers a DNA fragment (T-DNA) from an endogenous plasmid into the plant genome. For transfer of a gene of interest the gene is first inserted into the T-DNA region of Agrobacterium tumefaciens, which is subsequently used for infection using the floral dip method according to Clough and Bent, 1998 in Plant J 16: 735-743.

Antibody: immunoglobulin protein that is produced in the body in response to immunisation with an antigen (for example MKS1 polypeptide or peptide fragment thereof), and that binds specifically to that antigen.

Breeding program: A breeding program encompasses the selection of progeny resulting from a sexual cross between parent plants. The sexual cross may be between defined parent plants or between a random population of parent plants. The progeny resulting from the cross are selected according to defined selection criteria including, but not limited to agronomic performance e.g. disease resistance, drought resistance, heat tolerance, yield, and the inheritance of a specific gene including a transgene.

cDNA: complementary DNA, comprising a 1^{st} strand, complimentary to a mRNA molecule generated by reverse transcription, from which a 2^{nd} complementary strand may be generated with a polymerase.

Chimeric gene: refers to a nucleic acid sequence, comprising a promoter operably linked to a second nucleic acid sequence containing an ORF or fused ORFs, which optionally may be operably linked to a terminator sequence. The promoter sequence is not normally operatively linked to the second nucleic acid sequence as found in nature, but is able to regulate transcription or expression of the second nucleic acid sequence. The second nucleic acid sequence codes for a mRNA and may be expressed as a protein.

Conservatively modified variant: refers to a polypeptide sequence when compared to a second sequence, and includes individual conservative amino acid substitutions as well as individual deletions, or additions of amino acids. Conservative amino acid substitution tables, providing functionally similar amino acids are well known in the art. The following five groups each contain amino acids that are conservative substitutions for one another:

I: valine (V), leucine (L), isoleucine (I), methionine (M);

II: phenylalanine (F), tyrosine (Y), tryptophan (W);

III: arginine (R), lysine (K), histidine (H), glutamine (Q);

IV: aspartic acid (D), glutamic acid (E), asparagine (N), glutamine (Q);

V: alanine (A), serine (S), threonine (T).

In addition, individual substitutions, deletions or additions which alter, add or delete a single amino acid or a small percentage of amino acids in an encoded sequence are also “conservatively modified variants”. When referring to nucleic acid sequences, conservative modified variants are those that encode an identical amino acid sequence, (in recognition of the fact that codon redundancy allows a large number of different sequences to encode any given protein); or conservative modified variant; or a conservative modified variant having deletions or additions of a single amino acid or a small percentage of amino acids in the encoded sequence.

Crop: a crop encompasses plant biomass generated by the growth of a seed or seedling, and includes reproductive parts, e.g. seed, caryopsis, cob, or fruit and vegetative parts, e.g. leaf and tuber.

Dicotyledenous plant: flowering plant having two cotyledons in the seed.

Disease resistance: the term disease resistance indicates the ability of a plant to resist pathogen attack. As used herein “enhanced” resistance is a greater level of resistance to a disease causing pathogen by a transgenic or genetically modified plant, produced by the method of the present invention, as compared with a non-modified, control plant. In a preferred embodiment the level of resistance to a pathogen is at least 5%, preferably at least 10%, more preferably at least 20% greater than the resistance of a control plant.

Exon: protein coding sequence of a gene sequence.

Expression cassette: a nucleic acid sequence capable of directing expression of a particular nucleotide sequence in an appropriate host cell, comprising a promoter operably linked to the nucleotide sequence of interest, which is operably linked to termination signals. The expression cassette comprising the nucleotide sequence of interest may be chimeric, meaning that at least one of its components it heterologous with respect to at least one of its other components. The expression cassette may also be one which is naturally occurring but has been obtained in a recombinant form useful for heterologous expression. Typically, however, the expression cassette is heterologous with respect to the host, i.e., the particular nucleic acid sequence of the expression cassette does not occur naturally in the host cell and must have been introduced into the hosf cell or its progenitor by a transformation event.

Fusion protein: polypeptide read-through expression product of a gene comprising two or more protein coding sequences fused in frame.

Genetically modified plant: in terms of the present invention relates to a non-naturally occurring plant, whose genome has been artificially modified by genetic manipulation techniques, e.g., chemical mutagenesis, site-directed mutagenesis, homologous recombination (Terada et al. 2002 Nature Biotech. 20: 1030-1034) and transformation.

Genomic DNA: DNA sequences comprising the genome of a cell or organism.

Heterologous: a polynucleotide sequence is “heterologous to” an organism or a second polynucleotide sequence if it originates from a foreign species, or from a different gene, or is modified from its original form. A heterologous promoter operably linked to a coding sequence refers to a promoter from a species, different from that from which the coding sequence was derived, or, from a gene, different from that from which the coding sequence was derived.

Homologous: a polynucleotide sequence is “homologous to” an organism or a second polynucleotide sequence if it originates from the same species, or gene. A homologous promoter refers to a gene promoter operably linked to the coding sequence of the same gene.

Homologue: is a gene or protein that is substantially identical to another gene's sequence or another protein's sequence.

Host cell: A prokaryotic or eukaryotic cell which may be transformed with an expression casette cloned in a vector. The host cell may be a bacterial (for example Agrobacterium spp, or E. coli) or plant cell (for example a monocotyledenous or dicotyledenous plant cell. The protein encoded by the expression cassette may be expressed and purified from the host cell.

Identity: refers to nucleic acid or polypeptide sequences that are the same or have a specified percentage of nucleic acids of amino acids that are the same, when compared and aligned for maximum correspondence over a comparison window, as measured using one of the sequence comparison algorithms listed herein, or by manual alignment and visual inspection. When percentage of sequence identity is used in reference to proteins, it is recognized that residue positions that are not identical often differ by conservative amino acid substitutions, where amino acid residues are substituted for amino acid residues with similar chemical properties (e.g., charge or hydrophobicity) and therefore do not change the functional properties of the molecule. Where sequences differ in conservative substitutions, the percent sequence identity may be adjusted upwards to account for the conservative nature of the substitution. Typically this involves scoring a conservative substitution as a partial rather than a full mismatch, thus increasing the percent identity. Means for making these adjustments are well known to those skilled in the art.

Interacting: in terms of the present invention, relates to a physical interaction between two or more proteins, and their association for a duration sufficient to be detectable by known bioassays. For example, interacting proteins are detected by the yeast 2-hybrid screen and assay, and by co-precipitation with antibodies with affinity to one of the interacting proteins.

Intron: is a non-coding sequence interrupting a protein coding sequence within a gene sequence.

Isolated: in the context of the present invention an isolated protein (polypeptide) or an isolated nucleic acid molecule is a protein or nucleic acid molecule that, by the hand of man, exists apart from its native environment, and is therefore not the product of nature. The isolated protein or nucleic acid molecule may exist in a purified form or in a non-native environment such as, for example, a transformed host cell.

MAP kinase: mitogen-activated protein kinase, which acts downstream of other MAPK kinases, in reversible phosphorylation cascades to transduce extracellular signals into cellular responses (for example MPK4, 3, 5, 6, 17).

MKS1: MAP Kinase Substrate1 (MKS1) polypeptide is a positive regulator of SAR and enhances plant disease resistance. The primary amino acid sequence of MKS1 comprises domain 1 with sequence:

GPRPXPLSVXXDSHKIKKP and domain 2 with sequence:

PWIYXXSPKVVHXXXXEFMXWQRLTG, and conservatively modified variants thereof, wherein X refers to any amino acid residue. MKS1 is phosphorylated at one or more sites by a MAP kinase and it interacts with a transcription factor (for example a WRKY transcription factor). A MKS1 polypeptide includes a truncated or deleted fragment thereof that retains domain 1 and domain 2 sequences and the functional properties of being a positive regulator of SAR and enhancing plant disease resistance. Domain I has the functional property of comprising part or all of the interaction site for MAK kinase, while domain 2 has the functional property of comprising part or all of the interaction site for a transcription factor e.g. a WRKY-type transcription factor.

Monocotyledenous plant: includes, but is not limited to, barley, maize, oats, rice, rye, sorghum, wheat and members of the grass family Poaceae.

Mutant: a plant or organism with a modified genome sequence resulting in a phenotype which differs from the common wild-type phenotype.

Native: as found in nature, and with respect to “native promoter” refers to a promoter operably linked to its homologous coding sequence.

RNA blot analysis: a technique for the quantitative analysis of mRNA species in an RNA preparation involving size separation of RNA by agarose gel electrophoresis, subsequent transfer of RNA from the gel to a nucleic acid binding membrane, and hybridisation of the membrane with sequence specific probes.

Operably linked: refers to a functional linkage; for example between a promoter and a second sequence, wherein the promoter sequence initiates transcription of RNA corresponding to the second sequence.

ORF: Open Reading Frame, which defines one of three putative protein coding sequences in a DNA polynucleotide.

Orthologue: Homologous genes (or proteins) diverging concurrently with the evolutionary divergence of the organism harbouring them. Orthologues commonly serve the same function within the organisms and are most often located in a similar position on the genome.

PCR: Polymerase Chain Reaction is a technique for the amplification of a DNA polynucleotide, employing a heat-stable DNA polymerase and short oligonucleotide primers, which hybridise to the DNA polynucleotide template in a sequence specific manner and provide the primer for 5′ to 3′ DNA synthesis. Sequential heating and cooling cycles allow denaturation of the double-stranded DNA template and sequence-specific annealing of the primers, prior to each round of DNA synthesis. PCR is used to amplify a DNA polynucleotide employing the following standard protocol or modifications thereof:

PCR amplification is performed in 25 μl reactions containing: 10 mM Tris-HCl, pH 8.3 at 25° C.; 50 mM KCl; 1.5 mM MgCl₂; 0.01% gelatin; 0.5 unit Taq polymerase and 2.5 pmol of each primer together with template genomic DNA (50-100 ng) or cDNA. PCR cycling conditions comprise heating to 94° C. for 45 seconds, followed by 35 cycles of 94° C. for 20 seconds; annealing at X° C. for 20 seconds (where X is a temperature between 40 and 70° C. defined by the primer annealing temperature); 72° C. for 30 seconds to several minutes (depending on the expected length of the amplification product). The last cycle is followed by heating to 72° C. for 2-3 minutes, and terminated by incubation at 4° C.

Phosphorylated: in terms of the present invention relates to the phosphorylation of a protein, such as MKS1, by a protein kinase, such as a MAP kinase. Phosphorylation sites are commonly serine and/or threonine residues on the protein. Protein kinases act to regulate the activity of proteins by covalently attaching phosphate groups. The addition of this large charged group to the protein will usually result in changes in the target protein's conformation. These conformational changes typically result in changes in the protein's activity (either up or down) or association with other proteins. Protein phosphatases act in an opposite fashion and regulate proteins by removing phosphate groups that have been covalently attached to a protein (by a protein kinase).

Polynucleotide molecule: or “polynucleotide”, or “polynucleotide sequence” or “nucleic acid sequence” refers to deoxyribonucleotides or ribonucleotides and polymers thereof in either single- or double-stranded form. The term encompasses nucleic acids containing known analogues of natural nucleotides, which have similar binding properties as the reference nucleic acid.

Polypeptide: is any chain of amino acids, regardless of length or post-translational modification (for example glycosylation or phosphorylation).

Pathogenesis Related (PR) gene: is one that is activated or expressed in a cell of a plant in conjunction with pathogen attack and infection of the plant by a pathogen. Proteins encoded by PR genes include chitinase, extension (EXT1), PR1, PR5, Lipid transfer protein (LTP), β-1,3-glucanase (BGL2/PR2), β-1,3-glucanase (BGL3), glutathione-S-transferase (ERD11, PM24), LRR receptor kinase, monodehydroascorbate reductase, thionin, osmotic, glycine-rich protein (GRR), phenylammonialyase (PAL), oxalate oxidase-like (GKP5).

Promoter: is an array of nucleic acid control sequences that direct transcription of an operably linked nucleic acid. As used herein, a “plant promoter” is a promoter that functions in plants. Promoters include necessary nucleic acid sequences near the start site of transcription, e.g. a TATA box element, and optionally includes distal enhancer or repressor elements, which can be located several 1000 bp upstream of the transcription start site. A “tissue specific promoter” is one that specifically regulates expressed in a particular cell type or tissue, for example the promoter from the Arabidopsis thaliana RuBisCo small subunit gene NM_{—}179480 [gi:30695946]. A “constitutive” promoter is one that is active under most environmental and developmental conditions throughout the plant, for example the 35S CaMV promoter (Acc.No:V00141, J02048), the Arabidopsis and maize UBI1 gene promoter (Christensen et al., 1992, Plant Mol Biol 18: 675-689), maize ADH gene promoter (Last et al. 1991 Theor Appl Genetics 81: 581-588), rice ACT1 gene promoter (McElroy et al. 1990 Plant Cell 2: 163-172). An “inducible promoter” is one which is activated in the presence of a specific agent (the inducer), which may be a chemical compound or a physical stimulus such as heat or light. The chemical compound may be one that is not found in the plant in an amount sufficient to induce activation of the inducible promoter and transcription of the operably linked gene. Examples of inducible promoters include the ecdysone agonist inducible promoter (Martinez et al. 1999 Plant J. 19: 97-106), glucocorticoid agonist inducible promoter (Aoyama and Chua, 1997 Plant J. 11: 605-612), copper inducible promoter (Mett et al. 1993 Proc Natl Acad Sci USA 90: 4567-4571), ethanol inducible promoter (Caddick et al. 1998 Nature Biotech 16: 177-180), tobacco WUN1 promoter (Seibertz et al. 1989 Plant Cell, 1: 961-968) and the disease-inducible WRKY28 promoter (gi:17064157; Dong et al., 2003 Plant Mol. Biol., 51: 21-37), and an inducible MKS1 gene promoter may itself be used to direct expression in a MKS1 coding sequence.

RACE/5′RACE/3′RACE: Rapid Amplification of cDNA Ends is a PCR-based technique for the amplification of 5′ or 3′ regions of selected cDNA sequences which facilitates the generation of full-length cDNAs from mRNA. The technique is performed using the following standard protocol or modifications thereof: mRNA is reverse transcribed with RNase H⁻ Reverse Transcriptase essentially according to the protocol of Matz et al, (1999) Nucleic Acids Research 27: 1558-60 and amplified by PCR essentially according to the protocol of Kellogg et al (1994) Biotechniques 16(6): 1134-7.

Real-time PCR: a PCR-based technique for the quantitative analysis of mRNA species in an RNA preparation. The formation of amplified DNA products during PCR cycling is monitored in real-time, using a specific fluorescent DNA binding-dye and measuring fluorescence emission.

Recombinant vector: a DNA molecule comprising sequences allowing self-replication in one or more host cells, e.g. E. coli or Agrobacterium spp., which may further comprise a heterologous chimeric gene, inserted into the vector DNA molecule. A recombinant vector, comprising a chimeric gene, may be transformed into a host cell for the purposes of expressing the chimeric gene. A recombinant vector comprising a chimeric gene also encompasses vectors for transformation of a plant, for example binary vectors.

Regulator as referred to herein, is a protein which regulates another protein, pathway or response e.g. SAR, to either enhance or reduce the activity or level of said protein, pathway or response.

SAR: Systemic acquired resistance is a plant immune response which provides protection against a spectrum of pathogens in uninfected parts of a plant and is correlated with the expression of pathogenesis-related (PR) proteins, some with antimicrobial activity.

Sexual cross: refers to the pollination of one plant by another, leading to the fusion of gametes and the production of seed.

SMART consensus: represents the consensus sequence of a particular protein domain predicted by the Simple Modular Architecture Research Tool database (Schultz, J. et al. (1998)—PNAS 26; 95(11):5857-64)

Southern hybridisation: A filter carrying nucleic acid (DNA or RNA) is prehybridized for 1-2 hours at 65° C. with agitation in a buffer containing 7% SDS, 0.26 M Na₂HPO₄, 5% dextrane-suphate, 1% BSA and 10 μg/ml denatured salmon sperm DNA. Then a denatured, radioactively-labelled DNA probe is added to the buffer and hybridization is carried out over-night at 65° C. with agitation. Unbound and non-specifically bound probe is then removed from the filter by washing. For low-stringency hybridisation, washing is carried out at 65° C. with a buffer containing 2×SSC, 0.1% SDS for 20 minutes. For medium-stringency, washing is continued at 65° C. with a buffer containing 1×SSC, 0.1% SDS for 2×20 minutes, and for high-stringency filters are washed a further 2×20 minutes at 65° C. in a buffer containing 0.2×SSC, 0.1% SDS. Probe labelling by random priming is performed essentially according to Feinberg and Vogelstein (1983) Anal. Biochem. 132(1), 6-13 and Feinberg and Vogelstein (1984) Addendum, Anal. Biochem., 137(1), 266-267.

Substantially identical: refers to two nucleic acid or polypeptide sequences that have at least 60%, preferably 80%, most preferably 90-95% nucleotide or amino acid residue identity when aligned for maximum correspondence over a comparison window as measured using one of the sequence comparison algorithms given herein, or by manual alignment and visual inspection. This definition also refers to the complement of the test sequence with respect to its substantial identity to a reference sequence. A comparison window refers to any one of the number of contiguous positions in a sequence (being anything from between about 20 to about 600, most commonly about 100 to about 150) which may be compared to a reference sequence of the same number of contiguous positions after the two sequences are optimally aligned. Optimal alignment can be achieved using computerized implementations of alignment algorithms (e.g., GAP, BESTFIT, FASTA, and TFASTA in the Wisconsin Genetics Software Package, Genetics Computer Group, 575 Science Dr., Madison, Wis. USA) or BLAST analyses available on the site: (www:ncbi.nlm.nih.gov). Furthermore, substantially identical nucleic acid or polypeptide sequences perform substantially the same function.

Transcription factor: any protein required to initiate or regulate transcription of a gene, which may bind directly or indirectly to the DNA sequence of cis-elements of the gene (for example a WRKY transcription factor).

Transgene: refers to a polynucleotide sequence, for example a “chimeric gene”, which is integrated into the genome of a plant by means other that a sexual cross, commonly referred to as transformation, to give a transgenic plant.

Transgenic plant: a plant harbouring a transgene stably integrated into host DNA and inherited by its progeny.

UTR: untranslated region of an mRNA or cDNA sequence.

Wild type: a plant gene, genotype, or phenotype predominating in the wild population or in the germplasm used as standard laboratory stock.

III. Isolation of a MAP Kinase Substrate I Protein and its Homologues

The present invention concerns the protein MAP kinase substrate 1 (MKS1), isolated from Arabidopsis thaliana, and homologous or orthologous plant MKS1 proteins. As described more fully below in the examples, MKS1 is a positive regulator of the SAR signal transduction pathway, and plays a key role in the regulation of SA levels and PR gene expression in response to pathogen attack. MKS1 was identified by its interaction with MPK4, first detected in a yeast 2-hybrid screen. MPK4 is a negative regulator of SAR that represses SA-mediated defence responses (Petersen et al., 2000, supra). MKS1, isolated from Arabidopsis thaliana, is a polypeptide of 222 amino acids residues (Seq. ID No: 2; GI:18401970; At3g18690), having 12 putative phosphorylation sites. Interaction between MPK4 and MKS1 is further demonstrated to occur in vitro, and in vivo in Arabidopsis plants. Interaction between MPK4 and MKS1 can furthermore lead to phosphorylation of MKS1 at one or more phosphorylation sites, where phosphorylation of residue S30 has been confirmed. MKS1, expressed as a GFP-fusion protein, is co-localised in the nucleus of leaf mesophyll cells, together with MPK4. The targeting of MKS1, as well as MPK4, to the nucleus is consistent with its role in the SAR signal transduction pathway and induction of PR gene transcription. MKS1, isolated from Arabidopsis thaliana, is encoded by the intron-less gene (Seq ID No: 1; GI:18401969; At3g18690), whose function was previously unknown.

MKS1 is shown to interact with down-stream components of the SAR signal transduction pathway, which are involved in the regulation of PR gene expression. The transcription factors WRKY25 (Acc.No:GI:15991726) and WRKY33 (Acc.No:GI:21105639) are identified as interaction partners of MKS1 by 2-hybrid screening and directed 2-hybrid assay. These transcription factors are Group 1 members of a large family of WRKY plant transcription factors, which are characterised by a N-terminal WRKY domain having the conserved amino acid sequence WRKYGQK, together with a zinc finger motif (Eulgem et al. 2000, Trends in Plant Sci 5: 199-206). WRKY proteins bind to highly conserved cis-acting W box elements (T)(T)TGAC(C/T), which are present in defence response genes, including PR1. Although the evidence for a role of WRKY transcription factors in regulating plant defence responses is convincing, the function of the majority of members of the WRKY family is yet to be elucidated. The phosphorylation of MKS1 by MPK4, combined with the protein-protein interaction between MPK4 and MKS1 and between MKS1 and WRKY25 and 33, clearly establish MKS1 as a key regulatory protein in the SAR signal transduction pathway.

SAR is a plant defence mechanism, which is widely conserved in the plant kingdom (Dumer J. et al., 1997 Trends in Plant Science 2: 266-274). Thus MKS1 homologues, which function as regulator proteins in the SAR signal transduction pathway, may be found in other plants, including crop plants. MKS1 homologues and orthologues can be identified by a standard protein-protein BLAST or tblastn search against the database www: ncbi.nlm.nih.gov/blast/BLAST.cgi. Since the isolated Arabidopsis MKS1 is encoded by the MKS1 gene sequence At3g18690 (GI:18401969), an nblastn search may similarly be performed to identify plant genes encoding MKS1 homologues and orthologues. The application of this approach is illustrated in the Examples, where MKS1 homologues or orthologues are identified in Arabidopsis (Seq ID No: 6; At1g21326), Brassica oleracea (Seq ID No: 10 and 14), Glycine max (Seq ID No: 16), and Oryza sativa (rice) (Seq ID No: 20), encoded by MKS1 gene homologues or orthologues in Arabidopsis (Seq. ID. No: 5; GI:22329704), Brassica oleracea (Seq ID No: 9, GI:17796488, BoBH544707; Seq ID No: 13, BoBOHBT92TR+BOGQI24TF), Glycine max (Seq ID No: 15; GI:8283399, GmBE020960), and Oryza sativa (rice) (Seq ID. No: 19, OsCAD40925;), respectively. Additional MKS1 homologues or orthologues are found in rice (Seq ID No: 26, OsAP004654) maize (Zea mays) (Seq ID No: 27, ZmCC613160; Seq ID No: 28, ZmCC635639), tobacco (Nicotiana tabacum) and clover (Medicago truncatula) as exampled in FIG. 1B. In an alternative approach, nucleotide sequences encoding plant MKS1 homologues or orthologues can be identified in libraries constructed from plant genomic or cDNA by hybridisation screening with a polynucleotide probe comprising 20 or more consecutive nucleotides of an MKS1 gene (for example At3g18690). Hybridisation screening is performed according to standard protocols, under conditions defined above. Plant genomic or cDNA may also be screened for nucleotide sequences encoding plant MKS1 homologues or orthologues by PCR, using primer sequences comprising 15 or more consecutive nucleotides of an MKS1 gene (for example At3g18690), and a standard PCR amplification protocol as defined above. The PCR amplification of nucleotide sequences encoding MKS1 can also be performed using degenerate primers whose design is based on conserved amino acid sequences in MKS1, which can be identified by ClustalW alignment of MKS1 homologues or orthologues, as shown in the Examples. In the case that a MKS1 cDNA sequence is a partial sequence, the corresponding full-length MKS1 cDNA may be generated using 5′ and 3′ RACE as defined above.

A MKS1 protein homologue or orthologue is characterised by a primary sequence that comprises domain 1 with sequence:

IXGPRPXPLXVXXDSHXIKK and domain 2 with sequence:

PWIYXXSPKWHXXXXEFMXWQRLTG, [wherein X refers to any amino acid residue], or conservatively modified variants of said domain sequences. Domain 1 of MKS1 has the functional property of interacting with the MAP kinase (e.g. MPK4) and comprising a serine-proline phosphorylation site that is phosphorylated (e.g. by MPK4), consistent with its sequence homology to a MAPK docking site (Sharrocks et al., 2000, Trends Biochem. Sci. 25: 448-453). Domain 2 has the functional property of interacting with a transcription factor, in particular a WRKY-type transcription factor.

A MKS1 protein homologue or orthologue is substantially identical to a MKS1 protein with Seq ID No: 2, 6, 10, 14, 16, 20, 26, 27 or 28, furthermore comprising amino acid sequence domains 1 and 2, (given above) or conservatively modified variants thereof. A nucleic acid molecule encoding a MKS1 protein homologue or orthologue is characterised by a nucleotide sequence that is a substantially identical to a nucleic acid molecule with Seq ID No: 1, 5, 9, 13, 15 or 19, or more preferably a conservatively modified variant thereof. Furthermore, a MKS1 protein homologue or orthologue is characterised by the properties of being a positive regulator of SAR, enhancing plant disease resistance, being phosphorylated by a MAP kinase and interacting with a transcription factor regulating SAR gene expression, e.g. WRKY transcription factor. Phosphorylation of MKS1 by a MAP kinase can be detected by in vitro phosphorylation assay as illustrated in the Examples. Interaction of MKS1 with a transcription factor can be detected by yeast 2-hybrid screens and directed 2-hybrid assays as illustrated in the Examples.

IV Transgenic Plants with Modified Expression of MKS1 Protein

A nucleic acid molecule encoding MKS1 protein can be used to modify and enhance MKS1 protein expression in a transgenic plant of the invention and thereby induce a SAR response and increase the pathogen resistance in the plant. The MKS1 encoding nucleic acid molecule and the method provided by the invention can be utilised to induce SAR and confer disease resistance in a wide variety of plants. These plants include a monocotyledenous crop plant such as barley, maize, oats, rice, rye, sorghum and wheat; and a member of the grass family of Poaceae, such as Phleum spp., Dactylis spp., Lolium spp., Festulolium spp., Festuca spp., Poa spp., Bromus spp., Agrostis spp., Arrhenatherum spp., Phalaris spp., and Trisetum spp., for example, Phleum pratense, Phleum bertolonii, Dactylis glomerata, Lolium perenne, Lolium multiflorum, Lolium multiflorum westervoldicum, Festulolium braunii, Festulolium loliaceum, Festulolium holmbergii, Festulolium pabulare, Festuca pratensis, Festuca rubra, Festuca rubra rubra, Festuca rubra commutata, Festuca rubra trichophylla, Festuca duriuscula, Festuca ovina, Festuca arundinacea, Poa trivialis, Poa pratensis, Poa palustris, Bromus catharticus, Bromus sitchensis, Bromus inermis, Deschampsia caespitosa, Agrostis capilaris, Agrostis stolonifera, Arrhenatherum elatius, Phalaris arundinacea, and Trisetum flavescens.; and a dicotyledenous plant, such as alfalfa, carrot, cotton, potato, sweet potato, oilseed rape, radish, soybean, sugarbeet, sugar cane, sunflower, tobacco, turnip; vegetables such as asparagus, bean, carrot, chicory coffee, celery, cucumber, eggplant, fennel, leek, lettuce, garlic, onion, papaya, pea, pepper, spinach, squash, pumpkin, tomato; vegetable brassicas such as brussel sprouts, broccoli, cabbage, cauliflower; fruits, such as avocado, banana, blackberry, blueberry, grapes, mango, melon, nectarine, orange, papaya, pineapple, raspberry, strawberry; rosaceous fruits such as apple, apricot, peach, pear, cherry, plum and quince; herbs such as anise, basil, bay laurel, caper, caraway, cayenne pepper, celery, chervil, chives, coriander, dill, horseradish, lemon balm, liquorice, marjoram, mint, oregano, parsley, rosemary, sesame, tarragon and thyme; woody species, such as eucalyptus, oak, pine and poplar. The coding sequence of an MKS1 gene can be amplified by PCR using sequence specific primers, for example: Arabidopsis MKS1 (Seq ID No: 1) is amplified by the primer pair (Seq ID No: 3 & 4); Arabidopsis MKS1 (Seq ID No: 5) is amplified by the primer pair (Seq ID No: 7 & 8); Brassica oleracea MKS1 (Seq ID No: 9) is amplified by the primer pair (Seq ID No: 11 & 12); Oryza MKS1 (Seq ID No: 19) is amplified by primer pair (Seq ID No: 21 & 22). Glycine max MKS1 (Seq ID No: 15) comprises coding sequence for a part of MKS1 protein, and the complete MKS1 coding sequence may be generated by 5′ and 3′RACE, as described above using primers for 3′ extension (Seq ID. No: 17) and 5′ extension (Seq ID No: 18). An expression cassette is constructed comprising a nucleic acid sequence encoding a MKS1 polypeptide, substantially identical to protein SEQ ID No: 2, 6, 10, 14, 16 or 20 and furthermore comprising a domain I (IXGPRPXPLXVXXDSHXIKK) and domain 2 (PWIYXXSPKWHXXXXEFMXWQRLTG) amino acid sequence or conservatively modified variants thereof, wherein said nucleic acid sequence is operably linked to a heterologous or homologous promoter and 3′ terminator. The expression casette can be transformed into a selected host plant using a number of known methods for plant transformation. By way of example, the expression cassette can be cloned between the T-DNA borders of a binary vector, and integrated into an Agrobacterium tumerfaciens host by transformation, and used to infect and transform a host plant (Hinchee et al 1988 Bio/Technol. 6:915-922, Ishida et al., 1996 Nat. Biotechnol. 14:745-50). The expression cassette is commonly integrated into the host plant in parallel with a selectable marker gene giving resistance to an herbicide or antibiotic, in order to select transformed plant tissue. Stable integration of the expression cassette into the host plant genome is mediated by the virulence functions of the Agrobacterium host. Binary vectors and Agrobacteriurm tumefaciens-based methods for the stable integration of expression cassettes into the majority of all dicotyledenous and monocotylenous crop plants are known, as described for example for rice (Hiei et al., 1994, The Plant J. 6: 271-282) and maize (Yuji et al., 1996, Nature Biotechnology, 14: 745-750). Alternative transformation methods, based on direct transfer can also be employed to stably integrate expression cassettes into the genome of a host plant, as described by Miki et al., 1993, “Procedure for introducing foreign DNA into plants”, In: Methods in Plant Molecular Biology and Biotechnology, Glick and Thompson, eds., CRC Press, Inc., Boca Raton, pp 67-88). Promoters to be used in the expression cassette of the invention include constitutive promoters, for example the 35S CaMV promoter (Acc.No:V00141, J02048), the Arabidopsis and maize. UBI1 gene promoter (Christensen et al., 1992, Plant Mol Biol 18: 675-689), maize ADH gene promoter (Last et al. 1991 Theor Appl Genetics 81: 581-588), rice ACT1 gene promoter (McElroy et al. 1990 Plant Cell 2: 163-172). In an alternative embodiment, an inducible promoter may be used in the expression cassette to direct MKS1 expression. Examples of suitable inducible promoters include the ecdysone agonist inducible promoter (Martinez et al. 1999 Plant J. 19: 97-106), glucocorticoid agonist inducible promoter (Aoyama and Chua, 1997 Plant J. 11: 605-612), copper inducible promoter (Mett et al. 1993 Proc Natl Acad Sci USA 90: 4567-4571), ethanol inducible promoter (Caddick et al. 1998 Nature Biotech 16:177-180), tobacco WUN1 promoter (Seibertz et al. 1989 Plant Cell, 1: 961-968) and the disease-inducible WRKY28 promoter (gi:17064157; Dong et al., 2003 Plant Mol. Biol., 51: 21-37). Additionally, the inducible MKS1 gene promoter may itself be used to direct expression in the MKS1 expression cassette. An example of a suitable tissue-specific promoter includes the promoter from the Arabidopsis thaliana RuBisCo small subunit gene NM_{—}179480 [gi:30695946]. A terminator that may be used in the expression construct can for instance be the NOS terminator (Acc No: NC_{—}003065) (SEQ ID No: 24), the terminator of the Arabidopsis thaliana RuBisCo small subunit gene NM_{—}179480 [gi:30695946]. The recombinant vector comprising the MKS1 expression cassette is optionally transformed into a plant cell together with a selectable marker gene which is located on the same or a separate recombinant vector. Marker genes that facilitate selection of transformed plant cells, may encode peptides providing resistance to herbicide, antibiotic or drug resistance, for example, resistance to protoporphyrinogen oxidase inhibitors, hygromycin, kanamycin, G418, gentamycin, lincomycin, methotrexate, glyphosate, phosphinothricin. Optionally, host plants transformed with an expression cassette encoding a MSK1 protein, can be crossed with a second non-transgenic plant and progeny expressing said MKS1 protein can then be selected and used in the invention.

Transgenic plants comprising a transgene expressing a MKS1 polypeptide can be used in a breeding program, in order select plants with enhanced agricultural performance that have inherited the transgene. Transgenic plants as well as plant progeny selected in such a breeding program may be cultivated for the purpose of harvesting a crop, where the crop may be vegetative plant parts, e.g. leaf or tuber, or reproductive parts including seed, caryopsis, cob or fruit.

V Plant Disease Resistance of Transgenic Plants with Modified MKS1 Expression

The expression of MKS1 in transgenic plants, transformed with a MKS1 expression cassette, will be determined by the promoter to which the MKS1 coding sequence is operably linked. Where MKS1 expression is placed under the control of a constitutive promoter, MKS1 will be expressed throughout the plant at all developmental stages. The expression pattern of MKS1 will in turn determine the SAR response pattern in the plant and the level of resistance to plant pathogen attack. Since MKS1 induces SA synthesis, all basal pathogen resistance mechanisms induced by SA will be up-regulated by MKS1 expression. Since MKS1 does not regulate expression of jasmonate-induced genes, its expression in transgenic plants will not impair jasmonate-dependent wound responses in a plant. Furthermore, since MKS1 appears to act upstream of NPR1 in the SAR signal transduction pathway, it is expected to regulate a broader range of disease responses in a plant. Methods for assessing plant pathogen resistance are well known (Jach et al. 1995 Plant J. 8: 97-109; Whalen et al. 1991 Plant Cell 3: 49-60), and may be adapted according to the principal pathogens of the transgenic plant species. One method for assessing the resistance of a transgenic Arabidopsis plant, transformed with a MKS1 expression cassette, to a bacterial pathogen (Pseudomonas syringae) attack is given in the Examples. Other methods for evaluating disease resistance in plants are described by Crute et al 1994, Arabidopsis, Cold Spring Harbor Press, pp 705-747. Other examples of plant pathogens include the bacterial pathogens, Erwinia (for example E. carotovora), Xanthomonas (for example X. campestris and X. oryzae). Examples of fungal or fungal-like disease causing pathogens include Alternaria, Ascochyta, Botrytis, Cercospora, Colletotrichum, Diplodia, Erysiphe, Fusarium, Gaeumanomyces, Helminthosporium, Macrophomina, Nectria, Perenospora parasitica, Phoma, Phymatotrichum, Phytophora, Plasmopara, Podosphaera, Puccinia, Puthium,Pyrenophora, Pyricularia, Pythium, Rhizoctonia, Scerotium, Sclerotinia, Septoria, Thielaviopsis, Uncinula, Venturia and Verticillium.

The level of SAR in the transgenic plant can also be assessed by measuring the level of SA in the transgenic plant leaves, and the level of PR gene induction. Steady-state levels of PR mRNA can be quantitated by RNA blot hybridisation or alternatively by real-time PCR, as defined above. Application of these methods to the detection and quantitation of SAR in transgenic plants expressing MKS1 constitutively is illustrated in the Examples.

VI Isolated MKS1 and Specific MKS1 Antibodies

A nucleic acid molecule encoding the MKS1 protein can be operably linked to a promoter sequence to form a chimeric gene capable of directing expression of the MKS1 protein in a host cell. The nucleic acid molecule encoding MKS1 protein (ORF) can be fused in frame with a nucleic acid sequence encoding a tag. The expression of MKS1 as a fusion protein comprising a tag (e.g. 6× histidine tag, or a glutathione-S-transferase tag) facilitates the purification of the expressed MKS1 protein. Affinity purification of tagged protein is well known in the art, and its application to the purification of MKS1 protein is described in the Examples. The chimeric gene can be cloned, as an expression cassette, in a recombinant vector, and transformed into a host cell. The expression cassette can be transformed into a bacterial cell e.g. E. coli and expression of tagged MKS1 protein can be controlled by an inducible promoter system, e.g. IPTG inducible promoters. Alternatively, an expression cassette can be transformed into a host plant cell, and transformed plants comprising the expression cassette can be selected. Protein extracts, prepared from tissue of the transformed plant expressing tagged-MKS1 protein, can be used for the affinity purification of tagged-MKS1.

Tagged-MKS1, MKS1, or peptide fragments thereof, can be used for the production of specific polyclonal and monoclonal antibodies. Synthetic peptides having amino acid sequence identity to 10 or more consecutive amino acid residues of a MKS1 protein can be synthesised and used as antigen for the production of specific MKS1 antibodies. It is common to couple the synthetic peptide to a carrier protein, e.g. PPD (Purified Protein Derivative; Bardarov et al. 1990, FEMS Microbiology Letters 71: 89-94), to enhance the stability of the antigen and improve the presentation of the antigen to the immune humoral response system. Polyclonal and monoclonal antibodies can be raised, screened and tested according to standard protocols, as given by Harlow and Lane (1988) Antibodies, A Laboratory Manual, Cold Spring Harbour Publ. NY. A variety of immunoassay formats may be used to select antibodies specifically immunoreactive for a protein. For example, solid-phase ELISA immunoassays, immunoblots, or immunohistochemistry are regularly used for this purpose. Typically a specific or selective reaction will be at least twice background signal or noise and more typically more than 10 to 100 times background.

### EXAMPLES

MPK4 is a plant protein kinase whose regulatory functions include default repression of SA-dependent SAR, a pathway that primarily mediates resistance to certain biotrophic pathogens via PR gene expression. In addition, MPK4 is involved in the activation of PDF1.2 expression in response to jasmonate and ethylene, pathways that mediate resistance against necrotrophs and wounding herbivores (Petersen et al., 2000 supra). Since the regulatory functions of MPK4 are dependent on its kinase activity, it is likely that MPK4 interacts with and phosphorylates protein substrates which directly or indirectly lead to the control of gene expression appropriate to various pathogen responses. Hence, the identification and isolation of a protein, which interacts with and is phosphorylated by MPK4, would provide a key regulator of SA-dependent SAR in plants.

### Example 1

### Arabidopsis MAP Kinase Substrate 1 (MKS1) Interacts with MPK4

A yeast two-hybrid screen was employed to identify proteins that interact with the MPK4 protein. The yeast two-hybrid screen, first described by Fields and Song in 1989 (Nature 340: 245-24) is a common method used to detect protein-protein interactions. This screen exploits inherent properties of transcription factors, namely that are composed of two separate domains: a DNA-binding domain and a transcription activation domain. A physical association of the two domains of a transcription factor is required in order for it to bind to a promoter and activate transcription of a downstream gene. DNA sequences encoding fusion proteins, comprising the DNA-binding domain or the activation domain of a transcription factor, can be constructed and co-expressed in yeast. Interaction between the two fusion proteins will result in a functional transcription factor. If a DNA binding domain-MPK4 fusion protein (bait), and an activation domain fused to an MSK1 interacting protein (prey) are simultaneously expressed in yeast, a functional interaction between the two fusion proteins can be detected by the transcription of nutritionally essential genes and reporter genes cloned in yeast. The yeast two-hybrid screen is commonly based on the detection of yeast colonies in which transcription of these essential genes enables cell growth on histidine- or leucine-deficient media, and detectable β-galactosidase activity.

An Arabidopsis cDNA library fused to the activation domain of a transcription factor (prey) was screened for potential MPK4 interacting partners using the following yeast two-hybrid system. Saccharomyces cerevisiae strain PJ69-4A (MATa trp1-901 leu2-3, 112 ura3-52 his3-200 gal4 gal80 LYS2::GAL1-HIS3 GAL2-ADE2 met2::GAL7-lacZ; (James et al, 1996, Genetics 144: 1425-1436) was used as host strain for two hybrid screening. Cells were grown at 30° C. in liquid YPD medium (www: clontech.com) or on YPD agar plates. Transformed yeast cells were grown in liquid SD medium or on SD agar (Minimal SD Agar Base; www:clontech.com) plates supplemented with drop-out supplements (www:clontech.com) lacking specific amino acids. Yeast cells were transformed using the lithium acetate/polyethylene glycol method (Ito et al., 1983, J Bacteriol 153: 163-168). Library screening was performed with the MPK4 bait encoded by the full-length MPK4 cDNA from Arabidopsis thaliana Ecotype Ler, cloned into the Bam H1 site in pGBD-C1 (James et a/1996, supra). Both GAL4-based library screens were performed with the Arabidopsis MATCHMAKER cDNA library cloned in pGAD10 GenBank #U13188 (www: clontech.com/techinfo/manuals). Two independent screens of the library were conducted with the MPK4 bait, and in total the number of screened clones (6×10⁷) covered the library 20 times. Subsequently 7.4 million colonies were screened with MKS1 as bait, corresponding to 25 times the number of individual clones in the library.

A single full-length cDNA, designated MAP Kinase Substrate 1 (MKS1), corresponding to the intron-less Arabidopsis gene At3g18690, was found to interact with MPK4 in the yeast two-hybrid screen, shown in FIG. 1A. A similar interaction was observed after switching MPK4 and MKS1 as prey and bait, respectively. To test the specificity of the MAP kinase interaction, the interaction of MKS1 with other plant MAP kinases was tested in the yeast two-hybrid assay. The following MAP kinase cDNA sequences were cloned as AD fusions in pGAD424 (www: clontech.com): MPK3 (nucleotides 149-1261 of NM_{—}114433) using BamHI/Sal1 sites; MPK5 (nucleotides 466-1218 in NM_{—}117204) using NcoI/Not1 sites; MPK6 (nucleotides 116-1303 in NM_{—}129941) using BamHI/Sal1 sites; MPK17 (nucleotides 1-1740 in NM_{—}126206) using NcoI/Not1 sites. In contrast to MPK4, the MPK3, 5, 6 or 17 (Ichimura et al. 2002, Trends in Plant Sci., 7, 301-308) preys did not interact with the MKS1 bait (FIG. 1A), confirming the specificity of the MKS1-MPK4 interaction.

MKS1 is a protein of 222 amino acid residues having a predicted molecular mass of 24 kDa, and the sequence of Seq ID No: 2 (At3g18690). MKS1 is encoded by nucleotides 80 to 748 of the Arabidopsis gene At3 g18690; GI: 18401970 (SEQ ID No: 1). MKS1 contains 11 putative MAP kinase phosphorylation sites (Ser-Pro), indicated in FIG. 1B, based on sequence homology to other described phosphorylation sites (minimal consensus sequence S/TP; Sharrocks et al., 2000, Trends in Biochem Sci., 25: 448-453). The coding sequence for MKS1 was used in a standard protein-protein BLAST and tblastn search against the database at the www:ncbi.nlm.nih.gov/blast/BLAST.cgi and www:arabidopsis.org/Blast sites. The BLAST searches identified the following nucleic acid sequences comprising ORFs coding for previously unknown proteins, now identified as: Arabidopsis MKS1 gene homologue (Seq ID No: 5; Acc.No:At3g21326) encoding MKS1 protein homologue (Seq ID No: 6), Brassica oleracea MKS1 gene homologue (Seq ID No: 9; Acc.No:BH544707; GI:17796488) encoding

MKS1 protein homologue (Seq ID No: 10), Brassica oleracea (Seq ID No: 13; Acc.No:BOHBT92TR+BOGQI24TF) encoding MKS1 protein homologue (Seq ID No: 14), Glycine max MKS1 gene homologue (Seq ID No: 15; Acc.No:BE020960) encoding MKS1 protein homologue (Seq ID No: 16), rice MKS1 gene homologue (Seq ID No: 19; Acc.No:CAD40925; GI: 21740554) encoding MKS1 protein homologue (Seq ID No: 20), rice MKS1 gene homologue (Acc.No:OsAP004654) encoding MKS1 protein homologue (Seq ID No: 26), maize MKS1 gene homologue (Acc.No:ZmCC613160) encoding MKS1 protein homologue (Seq ID No: 27), maize MKS1 gene homologue (Acc.No:ZmCC635639) encoding MKS1 protein homologue (Seq ID No: 28, which all share sequence identity with Arabidopsis MKS1 (Seq ID. No: 2) and comprise Domains 1 and 2, as shown in the protein alignment given in FIG. 1B. The alignment was generated with the aid of CLUSTAL programs (clustalw.genome.ad.jp; Jeanmougin, F. et al., (1998) Trends Biochem Sci, 23, 403-5; Thompson, J. D., et al. (1997) Nucleic Acids Research, 24:4876-4882; Higgins, D. G., et al. (1996) Using CLUSTAL for multiple sequence alignments. Methods Enzymol., 266, 383-402. 5) Thompson, J. D., Higgins, D. G. and Gibson, T. J. (1994) Nucleic Acids Research, 22:4673-4680; Higgins, D. G., et al. (1992) CABIOS 8,189-191; Higgins, D. G. and Sharp, P. M. (1989) CABIOS 5,151-153; Higgins, D. G. and Sharp, P. M. (1988) Gene 73,237-244). Furthermore, MKS1 homologues or orthologues are also found in maize (Zea mays), tobacco (Nicotiana tobacum) and clover (Medicago truncatula) (FIG. 1B). Arabidopsis MKS1 protein (At3g18690) shares a sequence identity of 84.8% with Brassica oleracea MKS1 (Acc.No:BH544707) and 78.4% with Brassica oleracea (Acc no: BOHBT92TR+BOGQI24TF). The identified MKS1 homologues all comprise amino acid sequence domains 1 and 2, or conservatively modified variants thereof.

### Example 2

### Arabidopsis MPK4 Interacts with and Phosphorylates MKS1 In Vitro

A. MPK4-MKS1 Interaction In Vitro

To substantiate the interaction between MPK4 and MKS1, detected in the yeast two-hybrid screen, in vitro interaction assays (pull-down assays) were performed with recombinant MPK4 and MKS1 proteins. Recombinant MKS1 was obtained by bacterial expression according to the following procedure. The full-length MKS1 coding sequence (At3g18690 nucleotides 80 to 748) was cloned in-frame with the glutathione-S-transferase (GST) gene in the Xho I site of pGEX-5× plasmid (www: amershambiosciences.com). Expression of the recombinant protein in E. coli BL21 (pLysS) cells (www:novagen.com) was induced with 0.1 mM isopropyl-β-D-thiogalactopyranoside (IPTG) at 30° C. for 3-4 h, and 2% ethanol was added before induction. GST protein was similarly expressed in E. coli from the pGEX-5× plasmid. GST and GST-fusion proteins were purified from whole cell extracts of E. coli by binding to glutathione-Sepharose 4B beads (www: amershambiosciences.com), in the presence of proteinase inhibitors (2 μg/ml leupeptin, 1 mM AEBSF (4-(2-Aminoethyl)-benzenesulfonylfluoride.HCl), 2 μg/ml antipain, 5 mM EDTA, 5 mM EGTA, 2 μg/ml aprotinin). Proteins used in pull-down assays were not eluted from the glutathione sepharose beads. ³⁵S-methionine-labelled MPK4 was generated by coupled transcription-translation of the bait plasmid pGBKT7-MPK4 from the two hybrid screen, using a T7 coupled reticulocyte lysate system (www: promega.com/tbs/tb126/tb126.pdf).

Pull-down assays were preformed as follows: 10 μl ³⁵S-MPK4 was mixed with 200 μl % BSA in Bead Binding (BB) Buffer (BB Buffer; 50 mM KPO₄ pH 7.5, 150 mM KCl, 1 mM MgCl₂, 2 μg/ml leupeptin, 1 mM AEBSF, 2 μg/ml antipain, 2 μg/ml aprotinin), incubated on ice for 15 min, and then centrifuged for 10 min at 4° C. The supernatant was added to 2-5 μg GST or GST-fusion protein bound to sepharose beads in 200 μl, 1% BSA in BB Buffer and incubated for 2 hrs at 4° C. with rotation. The beads were washed 3 times with 1 ml wash buffer (50 mM KPO₄, pH 7.5, 150 mM KCl, 1 mM MgCl₂, 10% glycerol, 5% Triton X-100) with proteinase inhibitors and were then subjected to SDS-PAGE (Sodium dodecyl sulphate polyacrylamide gel electrophoresis; Laemmli, 1970 Nature 227: 680) separation on 15% gels.

The pull-down assay demonstrated that in vitro synthesized MPK4 (FIG. 2A, lane 1) interacts with and was bound by recombinant MKS1-GST (Lane 3), but not by GST alone (Lane 2), thereby confirming the MPK4-MSK1 interaction detected in the yeast two-hybrid screen.

B. MPK4 Phosphorylation of MKS1 In Vitro

The ability of MPK4 to phosphorylate putative MAP kinase Ser-Pro phosphorylation sites in MKS1 was investigated by in vitro phosphorylation assays. Full-length and C-terminally truncated histidine-tagged MSK1 were expressed and purified from E. coli. MKS1 nucleotide sequence (nucleotides 80 to 748 of At3g18690) encoding full-length MKS1 protein, was cloned into the Xho I site of the pET15b plasmid (www:novagen.com). Nucleotide sequences encoding MKS-1 with terminal deletions, C1-C3, were constructed by restriction digest of the MKS1-HIS containing pET15b vector. The C1 deletion was generated with BstBI and Bpu1102I, the C2 deletion with NheI and Bpu1102I, and the C3 deletion with StyI and Bpu1102. The digested plasmids were end-filled by incubation with 3 U Klenow enzyme and 10 μM deoxyribonucleotides for 30 minutes at 37° C., and then religated by overnight incubation with ligase enzyme at 16° C. The plasmid constructs encoding full-length MKS1 (amino acids 1-222), C1-MKS1 truncation (amino acids 1-196), C2-MKS1 truncation (amino acids 1-123) and C3-MKS1 truncation (amino acids 1-73), as shown in FIG. 1B, were transformed into E. coli BL21 (pLysS) cells (www: novagen.com), expression was induced with 0.1 mM isopropyl-β-D-thiogalactopyranoside (IPTG) at 30° C. for 3-4 h, adding 2% ethanol prior to induction. Expressed MKS1 was extracted using BugBuster and Benzonase assisted protein extraction, and purified by affinity binding of the histidine tag to Ni-NTA resin, according to the instructions of the manufacturer (www:novagen.com).

HA (influenza hemagglutin antigen)-tagged MPK4 (HA-tag is 6× YPYDVPDYA) was expressed by transgenic Arabidopsis plants (Petersen et al., 2000, supra). The HA-MPK4 was purified from protein extracts of the plants as described (Romeis et al., 1999, Plant Cell 11: 273-287), except that a buffer change was not made prior to immunoprecipitation (Romeis et al., 1999, Plant Cell 11: 273-287). 100 μg of total protein was immunoprecipitated from the plant protein extract with 2 μg/ml monoclonal 12CA5 HA-antibody (Boehringer) by affinity to the HA tag. Protein concentrations were determined with the Bradford dye-binding procedure (Bradford, 1976, Anal Biochem 131: 248-254). The resulting sepharose beads, with immunoprecipitated MPK4, were washed in kinase buffer (200 μM ATP, 80 mM Tris-HCl, pH 7, 5, 8 mM EGTA, 120 mM MgCl₂, 4 mM Na₃VO₄, 4 mM DTT) to remove the immunoprecipitation buffer and suspended, as a 50% slurry, in kinase buffer.

Phosphorylation assays were performed by mixing 10 μl MPK4-sepharose slurry, 5 μg substrate protein and 0.4 μl 300 μM ³²P-γ-ATP (3 μCi) with kinase buffer in a final volume of 30 μl. The assay samples were incubated for 1 h with agitation at 30° C., where after the assay proteins were separated by SDS-PAGE, and the gels subsequently dried on Whatmann 3MM paper and the radiolabelled proteins detected on a phosphorimager screen.

HA-tagged MPK4, immunoprecipitated from Arabidopsis plants, is shown to in vitro phosphorylate MKS1 as efficiently as myelin basic protein (MBP; Sigmasource), which is a standard MAP kinase substrate, as shown in FIG. 2B, lanes 1 versus 5). Immunoprecipitated extracts of non-transgenic Arabidopsis plants (wt) failed to phosphorylate MKS1 (FIG. 2B, lanes 6-8) confirming that the HA-antibody specifically immunoprecipitates HA-tagged MPK4. Furthermore, a mutant HA-tagged MPK4, with substitutions in the kinase activation loop abolishing MPK4 activity (T201A/Y203F; Petersen et al., 2000, supra) was similarly found not to phosphorylate MKS1 or MBP.

In order to identify which sites in MKS1 are phosphorylated by MPK4, C-terminal MKS1 truncations (C1, C2, C3), lacking some of the putative Ser-Pro phosphorylation sites (FIG. 1B), were tested in the phosphorylation assay. HA-tagged MPK4 readily phosphorylated both full-length and C-terminal MKS1 truncations, including C3 MKS1, which retains only 2 putative phosphorylation sites (Ser30 and Ser72), as seen in FIG. 2B, lanes 2-4. In order to map the functional phosphorylation sites in the C3 MKS1 protein, the encoded MSK1 sequence was altered from Ser30 to Ala30 (S30A) by in vitro mutagenesis, by substituting the codon TCA for GCA in the full-length and C3 truncated MKS1 gene. Although the mutant C3 truncated MKS1 (C3-S30A) was not phosphorylated by HA-tagged MPK4, the mutant full-length MKS1 (S30A) was phosphorylated (FIG. 2C, lanes 1 and 2 versus 3 and 4). This indicates that MPK4 phosphorylates MKS1 at Ser30, as well as other additional sites in the MKS1 protein.

A synthetic 22 amino acid peptide (Pep22), corresponding to amino acid residues 13-35 of MKS1 and comprising Ser30, shown as in FIG. 1B, was synthesized by K J Ross (www:tagc.com). Pep22 is an efficient competitor of full-length MKS1 for phosphorylation by MPK4, when added to the in vitro assay in a molar ratio of 1:1 (Pep22:MKS1) as shown in FIG. 2D, top. The Flg22 peptide, with amino acid sequence QRLSTGSRINSAKDDMGLQIA, which is known to activate immediate pathogen responses via the flagellin receptor, involving MPK3, 5, 6 and 17 as well as WRKT 22 and 29 (Asai et al., 2002, Nature 415: 977-983), was used as a control in this assay. Since the FIG. 22 peptide did not compete MKS1 phosphorylation (FIG. 2D, bottom) it is likely that the Pep22 domain of MKS1 specifically interacts with MPK4.

C. MPK4 Interacts with the N-Terminal Region of MKS1

The location and sequence specificity of the interaction between MPK4 and MKS1 was investigated employing the yeast 2-hybrid system described in Example 1. Nucleic acid molecules encoding the three C-terminal truncated forms of MKS1, (see Example 2B) as well as an N-terminal truncated form, N1-MKS1 (amino acids 55-222; deleted region is indicated as N1—in Table 1) were cloned into the 2-hybrid vectors and screened as MPK4 interacting partners. Only the N-terminally deleted MKS1 failed to interact with MPK4, confirming that the N-terminal 54 amino acid region is essential for MPK4-MKS1 interaction. N-terminal amino acid residues in MKS1 that are essential for this interaction were examined by testing the ability of MPK4 to interact in the 2-hybrid system with mutant MKS1 polypeptides. Site-directed mutagenesis was used to generate nucleic acid molecules encoding 18 full-length mutant MKS1 polypeptides each having a single amino acid substituted with alanine (denoted ‘A’ in Table 1), localised in the N-terminal domain 1 and pep22 regions.

TABLE 1

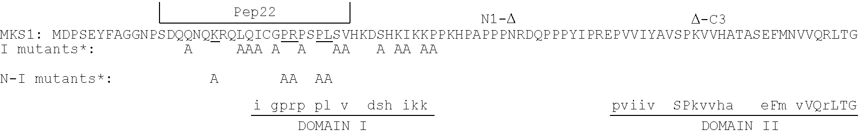

[*MKS mutants I (interactive) or N-I (non-interactive) with MPK4]

Mutation of five amino acid residues in the N-terminal region of MKS1, corresponding to the pep22/domain 1 region was found to prevent the interaction between MKS1 and its kinase MPK4. This domain 1, essential for MPK4 interaction, shares amino acid sequence homology with MAPK docking sites (Sharrocks et al., 2000, Trends Biochem. Sci. 25: 448-453).

### Example 3

### Antibodies for MKS1 Detection

To provide tools for the detection of MKS1 expression in vitro or in vivo in single or multicellular organisms, polyclonal (pa-Pep22) and monoclonal antibodies (ma-Pep22 & ma-Pep22p) were raised against the peptide Pep22 (SDQQNQKRQLQICGPRPSPLSVH), corresponding to amino acid residues 13-35 of MKS1. Ten to twelve-week old female Balb/cCF1 F1-hybrid mice were used to raise both polyclonal and monoclonal antibodies. The mice were primed with 0.2 mL live BCG vaccine, delivered intraperitoneally. One month later the mice were immunised with the antigen Pep22 coupled to PPD (Purified Protein Derivative; Bardarov et al. 1990, FEMS Microbiology Letters 71: 89-94), absorbed onto the adjuvant Al(OH)₃. The total volume of vaccine per immunisation was 500 μL, containing 15 μg of PPD and 1 mg of adjuvant. The antigen was injected intraperitoneally at 2-week intervals. To prepare polyclonal antibodies from the immunised mice, blood samples were collected 10 days after each immunisation and assayed for specific recognition of HIS-tagged MKS1 protein, expressed and purified from E. coli, followed by SDS-PAGE separation and semi-dry transfer and immunoblotting (Current protocols, www:wiley.com). Western blots were developed using alkaline phosphatase conjugated anti-mouse antibody (Promega). Monoclonal antibodies were prepared from immunised mice found to produce positive antisera, essentially as described by Kohler and Milstein (1975) in Nature 256: 495-497, as modified by Reading (1982) in J Immunol Methods 53: 261-291. After hybridoma cell fusions, culture supernatants were tested for specific recognition of HIS-tagged MKS1 protein, by enzyme-linked immunoabsorbent assay (ELISA; Current protocols, (www: wiley.com) and immunoblotting as described above for polyclonal antibodies.

Polyclonal antibody, pa-Pep22, specifically recognised MKS1 present in extracts of E. coli and wild type Arabidopsis plants, as shown by Western blotting in FIG. 3A. The same result was obtained using the monoclonal antibody ma-Pep22 (not shown). Monoclonal antibody ma-Pep22 (HYB 330-01), specifically recognised and immunoprecipitated MKS1 present in extracts of wild type Arabidopsis plants, since the immunoprecipated MKS1 was detected by the pa-Pep22 polyclonal antibody, as seen in lane 1 (upper band) of a Western blot (FIG. 3B). The lower band is due to binding of the secondary anti-IgG antibody to the ma-Pep22 light chain, which was also present in a control immunoprecipitation with ma-Pep22 where plant extract is omitted, seen in lane 2 (FIG. 3B).

### Example 4

### Arabidopsis MPK4 Interacts with MKS1 In Vivo

Interaction between MKS1 and MPK4 in vivo in Arabidopsis plants was demonstrated by the ability of the MKS1 specific monoclonal antibodies to co-immunoprecipitate MPK4 with MKS1 from leaf extracts. Leaf protein extracts were prepared as described in Example 2B, from transgenic mpk4 plants complemented to wild type by a functional HA-tagged MPK4 gene (FIG. 3C; Petersen et al., 2000, supra). Immunoprecipitates of the leaf extracts were analysed by SDS-PAGE and Western blots, which were probed with anti-HA antibody to detect HA-tagged MPK4. As shown in FIG. 3C lane 1, ma-Pep22 monoclonal antibody co-immunoprecipitated HA-tagged MPK4, which was detected with the anti-HA antibody. The co-immunoprecipitated HA-tagged MPK4 co-migrated with MPK4 immuno-detected in whole plant extracts (lane 1 versus lane 3). Monoclonal antibody (ma-Con), that does not detect MKS1 in plant extracts, was unable to immunoprecipitate MPK4 (lane 2). The upper bands immunodetected in lanes 1, 2 and 4 of FIG. 3C are likely due to binding of the secondary anti-IgG to the heavy chain of the immunoprecipitating monoclonal IgGs. Only the upper band was detected in a mock-plant extract containing MaPep22 (lane 4).

The in vivo phosphorylation status of MKS1 in extracts wild-type Arabidopsis leaves (Ler) as compared with mpk4 leaves, was examined following immunoprecipitation of MKS1 with ma-Pep22 monoclonal antibody. The MKS1 polyclonal antibody (pα-MKS1) detected equal amounts of MKS1 immunoprecipitated from wild-type and mpk4 plants, as shown in FIG. 3, D. However, a phosphoserine/phosphothreonine-specific antibody (α-pS/TP) only detected MKS1 in wild-type plants, thereby confirming that MKS1 is a substrate for MPK4 and is a key component of the signal transduction pathway in plants which facilitates a disease response.

### Example 5

### Transgenic Arabidopsis Plants with Modified MKS1 Expression

Transgenic plants, expressing elevated or reduced levels of MKS1 protein, were generated in Arabidopsis thaliana ecotype Landsberg erecta (Ler) via Agrobacterium-mediated transformation, according to the floral dip method (Clough and Bent, 1998 Plant J, 16:735-43). Transgenes were inserted between the T-DNA borders of pCAMBIA binary vectors, comprising the NPTII (kanamycin) resistance gene, and then transformed into Agrobacterium, and stably integrated into the Arabidopsis genome.

Constitutive over-expression of MKS1 in plants was obtained by the stable integration of CaMV 35S-MKS1 transgenes in Arabidopsis. The Arabidopsis MKS1 coding sequence (nucleotides 80-748 of Seq ID. No: 1 (At3g18690)) was amplified from its respective gene by PCR using a 5′ primer (Seq ID. No: 3) and 3′ primer (Seq ID No: 4). A transgene comprising the CaMV 35S promoter sequence (GI: 2173396; with Seq ID. No: 23), operably linked to a MKS1 coding sequence (nucleotides 80-748 of At3g18690), was generated by replacing the GUS ORF in pCAMBIA1301 (AF234297) by the MKS1 sequence, ligated with Nco I/Bst EII linkers. Arabidopsis transformants were selected by resistance to the antibiotic hygromycin incorporated into the seedling growth medium. Transformants with an integrated copy of the CaMV 35S-MKS1 transgene in the Arabidopsis genome were identified by northern blotting with a MKS1 probe (nucleotides 80-748 of At3g18690) and western blotting with maPep22.

Self-fertilisation of Arabidopsis transformants, with an integrated copy of the CaMV 35S-MKS1 transgene, led to seed formation, with the stable inheritance of the transgene in the progeny, and subsequent generations. Cross-pollination of the primary transformed plants or their progeny with control, non-transformed plants, generated progeny that inherited the transgene according to Mendelian genetics.

Silencing of MKS1 expression in plants by RNA interference (Chuang and Meyerowitz, 2000, Proc Natl Acad. Sci. USA. 97: 4985-4990) was obtained by the stable integration of a CaMV 35S-MKS1 RNAi transgene. The MKS1 coding sequence (nucleotides 80-748 of Seq ID No:1 (At3g18690)) was first inserted in the plasmid SLJ1382B1 (Andrea Ludwig and Jonathan D G Jones, Sainsbury Laboratory, UK), derived from plasmid SLJ4D4 (Jones et al. 1992, Transgenic Research 1: 285-297). The MKS1 coding sequence was cloned, in opposite orientations, on either side of an intervening intron in pSLJ1382B1, as 5′ Xba I/Fse I- and 3′ Asc I/Xho-linkered fragments. The intron had the sequence:

GTAAGTTTCTGCTTCTACCTTTGATATATATATATATTATCATTAATTAG TAGTMTATAATATTTCAATATTTTTTTCAAAATAAAAGAATGTAGTATAT AGCAATTGCTTTTCTGTAGTTTATAAGTGTGTATATTTTAATTTATAACTTT TCTAATATATGACCAAAATTTGTTGATGTGCAG and Seq ID. No: 25. The resultant RNAi cassette was excised with Eco RI and Hind III and cloned into corresponding sites in pCAMBIA3300 (derivative of pCAMBIA1201, AF234293). This construct was transformed into ecotype Col-0, and transformants were selected by resistance to spraying with the herbicide BASTA (Glufosinate-ammonium; www: bayercropscience.com).

Transgenic plants with elevated levels of MKS1, expressed under control of the constitutive promoter CaMV 35S (35S-MKS1), were identified by immunodetection of MKS1 in plant protein extracts analysed by western blotting with the polyclonal antibody pa-Pep22, a shown in FIG. 4A. Transgenic plants in which MKS1 expression was silenced by RNA interference were similarly identified by immunodetection of MKS1 levels in plant protein extracts (FIG. 4A). The 35S-MKS1 transgenic plants exhibited semi-dwarfism in contrast to the dwarf habit of mpk4 mutants (FIG. 4B). The MKS1-RNAi plants were phenotypically wild type in their growth habit (not shown).

### Example 6

### Properties of Transgenic Arabidopsis Plants with Modified MKS1 Expression

A. MKS1 Regulates the Expression of Pathogen Resistance Genes, But not Wound and Methyl Jasmonate Response Genes, in Plants

The steady-state levels of MKS1, PR and wound-induced gene transcripts were measured in total RNA samples extracted from Arabidopsis plants which were analysed by Northern blotting and hybridization with DNA probes according to standard protocols. DNA probes were amplified by PCR, with sequence-specific primers, from the following cDNA or genomic DNA templates: MKS1 (nucleotides 80-748 of At3g18690), PR1 (nucleotides 84-530 in M90508), PDF1.2 (EST 37F10T7), VSP (nucleotides 3-236 in ATTS0751/GBGA288), WR3 (WR3 probe in AtT5G50200, described by Leon J. et al., 1998, Mol Gen Gen 258: 412-419).

35S-MKS1 transgenic plants accumulated elevated levels of MKS1-mRNA compared to wild-type, consistent with increased MKS1 synthesis in these plants (FIG. 5A). Levels of the pathogen resistance PR1 mRNA, were enhanced in 35S-MKS1 transgenic plants and in mpk4 mutant plants when compared to wild-type plants (FIG. 5A, lane 1 versus 2 and 3).

Comparative transcript profiling of wild type, mpk4, and 35S-MKS1 plants was used to examine differences in global gene expression with the Affymetrix ATH1 array covering 22,810 transcripts. Total RNA was isolated from three replicates of wild type Ler, mpk4, and 35S-MKS1 grown in an chamber with 16 hr light (21C) and 8 hr dark (16C). RNA was amplified according to the standard Affymetrix protocol and hybridized to the Affymetrix ATH1 oligonucleotide microarray (Acc. # E-MEXP-173, ArrayExpress database, EBI). Raw intensity data was normalized using R implementation of qspline (Gautier et al., 2004, Bioinfomatics 20: 307-315). An implementation in the statistical language R of the logit-t method (Lemon et al., 2003, Genome Biol. 4: R67) applying one-way ANOVA was used to calculate statistical significance for differentially gene expression (R source code available on request). Genes with p-value less than 0.01 were considered significant. Fold change=max(over all j (median(Eij))−min(over all j (median(Eij)), where Eij is the ij'th gene expression index value, j is the genotype, and i the sample. Gene expression index values were calculated using the PM only implementation method (Li & Wong, 2001, Genome Biol. 2: R0032.1-11). Gene expression profiles for the 800 most significantly differentially expressed genes were clustered by k-means. Examples of over expressed genes in 35S-MKS1 are shown in Table 2.

In the 35S-MKS1 lines MKS1 was the most significantly differentially expressed gene due to its over-expression. Significantly, mRNAs of pathogen responsive genes including PR1, PR2 and the SA biosynthesis enzyme ICS1 were increased in both mpk4 and 35S-MKS1 plants as compared to wild-type.

TABLE 2

| Fold | Accession | Annotation | Fold | Accession | Annotation |
|---|---|---|---|---|---|
| 59.7 | At3g18690 | MKS1 | 29.7 | At4g01370 | MAP kinase 4 (MPK4) |
| 34.1 | At5g39100 | Germin-like protein (GLP6) | 21.2 | At1g53870 | Hypotheticql protein |
| 13.2 | At2g43570 | Endochitinase isolog | 20.6 | At5g44420 | antifungal protein (PDF1.2) |
| 11.1 | At2g14610 | PR1-like protein | 5.4 | At3g25760 | Hypothetical protein |
| 9.5 | At3g22600 | Lipid transfer protein | 4.3 | At2g36145 | Expressed protein |
| 8.1 | At1g75040 | Thaumatin-like protein (PR5) | 4.1 | At1g20190 | Expansin S2 precursor |
| 6.1 | At1g74710 | Isochorismate synthase (ICS1) | 3.9 | At1g21500 | Unknown protein |
| 5.3 | At1g69930 | Glutathione trnasferase | 3.8 | At4g26530 | Fructose bisphosphate aldolase |
| 4.2 | At3g57260 | Beta-1,3-glucanase (PR2) | 3.6 | At1g04800 | unknown protein |

MKS1, in contrast to MPK4, is shown not to be involved in the response to wounding and necrotrophic attack in plants. Plants respond to wounding and necrotrophic attack by the transcriptional activation of jasmonate and/or ethylene responsive genes including VSP, WR3 and PDF1.2, in which MPK4 is known to play a regulatory role (Petersen et al., 2000, supra; Andreasson E. and Mundy J, unpublished). The steady-state levels of these wound-induced genes was determined in Arabidopsis plants, subjected to wounding by making 1 to 3 cuts over the mid vein with a pair of scissors. VSP and WR3 mRNAs were induced in wild-type plants within 2 hours of wounding (FIG. 5B, lane 7), but were undetectable or greatly reduced in mpk4/NahG plants expressing the bacterial salicylate hydroxylase that degrades SA (FIG. 5B, lane 3). The same results were also seen following wounding of the mpk4 mutant (not shown). Silencing MKS1 expression in RNAi-MKS1 plants did not prevent a wild-type wounding response with the accumulation VSP mRNA (FIG. 5C, lanes 2 versus 4). These results indicate that MKS1 is not required for wound-responsive VSP expression. Silencing or over-expression of MKS1 did not significantly affect the levels of PDF2.1 mRNA accumulation following 48 hr of MeJA treatment (FIG. 5D). This indicates that MKS1, in contrast to MPK4 (Petersen et al. 2000 supra) is not required for MeJA responsive PDF1.2 expression.

B. Salicylic Acid Levels are Enhanced in 35S-MKS1 Transgenic Plants

The steady-state free and glycosylated salicylic acid content of Arabidopsis plants was analysed in plant extracts prepared by grinding plant tissue in liquid nitrogen, extracting the ground tissue in methanol, following by an ethylacetate:cyclopentane:isopropanol partition of the extract according to (Newman et al., 2001, Mol Plant-Microbe Interactions 14: 785-792). The salicylic acid content was analysed by HPLC using a diode array detector between 180-350 nm, as previously described Newman et al., 2001, supra. Salicylic acid levels were significantly elevated in 35S-MKS1 transgenic plants in comparison to wild-type plants, as shown in FIG. 6A.

C. Pathogen Resistance is Enhanced in 35S-MKS1 Transgenic Plants

Resistance to the plant pathogen Pseudomonas syringae is shown to be controlled by MKS1 expression levels in transgenic plants. Four-week-old Arabidopsis plants were infiltrated with a suspension of 1×10⁵ cfu/ml of virulent Pseudomonas syringae pv. tomato DC 3000 strain. Bacterial growth on infected plants was subsequently assayed by grinding four 0.5 cm² leaf pieces in 10 mM MgCl₂ for each sample. Dilutions were distributed on NYG agar plates containing rifampicin, cycloheximin and kanamycin, and colonies were counted, as previously described (Parker et al. 1996, Plant Cell 8: 2033-2046). 35S-MKS1 transgenic plants exhibited increased resistance to P. syringae DC3000, as seen for mpk4 plants, as shown in FIG. 6B. The disease response of 35S-MKS1 transgenic lines expressing different levels of MKS1, indicated that MKS1 expression is directly correlated with PR1 expression and resistance to Pseudomonas attack (data not shown). In contrast, of MKS1-RNAi plants were significantly less resistant to P. syringae DC3000 than wild type plants (FIG. 6C) confirming the key role of MKS1 in the development of SAR.

E. Localisation of MKS1 Expressed in Transgenic Plants

Green fluorescent protein (GFP) expressed in plant cells can be detected by virtue of its fluorescent properties, and GFP-protein fusions have provided a valuable tool for determining the whole plant and subcellular expression pattern of proteins of interest (Stewart, 2001, Plant Cell Rep. 20:376-82). Arabidopsis plants transformed with MKS1-GFP gene fusions, under control of CaMV 35S or MKS1 promoters, were generated to determine MKS1 cellular localisation. The MKS1 coding sequence with Eco RI linkers (nucleotides 80 to 748 in At3g18690) was N-terminally fused in frame with a GFP coding sequence, operably linked to a CaMV 35S promoter in the binary vector pCAMBIA 1302 (AF234297). The MKS1-GFP gene fusion cloned in pCAMBIA 1302 was placed under the control of the MKS1 promoter by substituting the CaMV 35S promoter by a 1.9 kb MKS1 promoter fragment (complement of nt 15531-13589 of BAC MVE11) having Nco I/Bst E11 linkers. The MPK4-GFP fusion was made by cloning a NotI linkered genomic fragment including 1150 bp promoter region from Ler genomic DNA cloned into pAVA393 (Amim et al, 1998 Gene 221: 3545). The control 35S-GUS-GFP fusion was included in the pCAMBIA1302, a derivative pCAMBIA1303. The transgenes in the binary vectors were transformed into Arabidopsis by Agrobacterium-mediated transformation and transgenic lines were selected as described in Example 5.

GFP expressed in mesophyll cells of young leaves of the transformed lines was visualised by confocal microscopy. GFP fluorescence was detected with a Zeiss LSM 510 laser-scanning microscope applying the 488 nm line of the argon laser and the corresponding dichroic mirror and a 505-530 nm band-pass filter. The generated images of GFP fluorescence in cells are vertical projections of variable numbers of optical sections.

The phenotype of the transgenic lines expressing the MKS1-GFP fusion protein was similar to that of the 35S-MKS1 transgenic lines expressing enhanced MKS1 levels. This indicates that MKS1 retains functional activity when expressed as a GFP-MKS1 fusion protein. Similarly, the MPK4-GFP fusion protein is functional and correctly targeted when expressed in transgenic plants, since it is able to complement the mpk4 mutant to wild type (Brodersen, Mattsson and Mundy, unpublished data). The GUS-GFP fusion protein, which lacks any specific subcellular, or extracellular targeting signals, was primarily localized to the cytoplasm of 35S GUS-GFP transgenic plants, as shown in FIG. 6D. GFP-MKS1, as well as GFP-MPK4, were localised in the nucleus of mesophyl cells, consistent with the demonstrated in vivo interaction of these two proteins and their transcriptional repression of downstream SAR effector genes under normal growth conditions (FIG. 6D).

In conclusion, transgenic plants with elevated levels of MKS1 expression show increased salicylic acid (SA) levels, PR gene expression and pathogen resistance, demonstrating that MKS1 is a key component of the SAR signal transduction pathway in plants controlling SAR and plant pathogen resistance. During negative regulation of SAR by MPK4 in wild-type plants, MKS1 is presumably phosphorylated, at one or more sites.

E. MKS1 Acts Down-Stream of MPK4 in the SAR Signal Transduction Pathway

MKS1-RNAi Arabidopsis plants, in which MKS1 expression is down-regulated, were crossed with mpk4 mutants to provide double mutants with the genotype MKS1-RNAi/mpk4. The mpk4 phenotype was partially suppressed in the double mutants, whereby the dwarf phenotype was reduced (FIG. 7a), the level of PR gene induction was reduced (FIG. 7b) and the plants were less resistant to pathogen attack (FIG. 7c). The ability of MKS1-RNAi genotype to suppress the mpk4 phenotype confirms the essential role of MKS1, downstream of MPK4, in the SAR signal transduction pathway.

### Example 7

### Arabidopsis MKS1 Interacts with WRKY 25 and 33 Transcription Factors

MKS1 is shown to be a key component of the SAR signal transduction pathway in plants, whose overexpression enhances SA levels and PR gene expression. The regulatory role of MKS1 is likely to be mediated by interaction with additional down-stream members of the pathway, including transcription factors. A yeast two-hybrid screen, with MKS1-BD as the bait, was used to identify proteins capable of interaction with MKS1. The MKS1-BD fusion was constructed by inserting the full-length MKS1 coding sequence (nucleotides 80-748 of At3g18690) into the Nco I restriction site of pGBKT7, and transformed into S. cerevisiae strain PJ69-4A (www: clontech.com). A GAL-4 based library screen in yeast of Arabidopsis MATCHMAKER cDNA libraries was performed as described in Example 1. 7. 4 million colonies were screened with the MKS1 bait, corresponding to 25 times the number of individual clones in the library. Two MKS1 interactors, the transcription factors WRKY25 (GI:15991725) and WRKY33 (GI:21105638), were identified in this screen, as shown in FIG. 1A. WRKY 33 and 25 are among the 70, or more, WRKY transcription factors predicted in Arabidopsis, which show amino acid sequence similarity and both belong to the group I WRKYs (Eulgem et al 2000 supra). Five different truncated WRKY33 proteins interacted with MKS1 in the yeast library, the shortest corresponding to the C-terminal 188 amino acids of WRKY33. This region comprises the C-terminal WRKY domain and a region denoted the A-motif (Eulgem et al., 2000, Trends in Plant Sci 5: 199-206).

The specificity of MKS1 interaction with WRKY transcription factors was examined in directed yeast two-hybrid assays with WRKY26 and WRKY29. Full-length cDNA WRKY26 (AF224699, nucleotides 23-949) and WRKY29 (AF442394, nucleotides 1-915) was fused with the nucleotide sequence encoding an AD domain in pGADT7 (www: clontech.com) using Bam H1 sites. WRKY26, of unknown function, is the next closest homolog to WRKY25 and 33, while the less similar WRKY29 positively regulates innate immunity responses involving MPK3 and 6 (Asai et al. 2002, Nature 415: 977-983). However, neither WRKY26 nor WRKY29 interacted with MKS1 in this assay (FIG. 1A), indicating that the interaction of MKS1 with WRKY25 and 33 is specific. No activity of the reporter His3, Ade2 or lacZ gene products was detected when any fusion protein construct was co-transformed with the corresponding empty vectors (data not shown).

The domain in the MKS1 polypeptide required for interaction with W33 was analysed in the yeast 2-hybrid system employing vector constructs expressing the N1-MKS1, C1-MKS1, C2-MKS1 and C3-MKS1 truncated polypeptides. Since on the C3-MKS1 failed to interact with W33 in yeast this localised the interaction domain between MKS1 and W33 to the conserved domain 11.

MKS1 is shown to be a positive regulator in the SAR signal transduction pathway, interacting with the MAP kinase MPK4 and the transcription factors WRKY25 and WRKY33. The defence response pathways, triggered by pathogen attack or wounding, which involve a series of signalling steps controlled by regulator proteins leading to the expression of resistance genes, are outlined in a model presented in FIG. 7. It is proposed that the negative regulator MPK4 represses SAR by phosphorylating and interacting with MKS1 to form a complex. The MPK4-MKS1 complex may, in turn, phosphorylate the transcription factors WRKY25 and 33 that may repress transcription of a salicylic acid promoter factor. The interaction of WRKY factors with promoters (W box motifs) is known to be phosphorylation dependent (Eulgem et al. 2000, supra).

MKS1 is a key regulatory protein of plant SAR and thereby controls the ability of plants to survive pathogen attack. Transgenic plants expressing enhanced levels of MKS1 protein show a significantly increased level of disease resistance. Thus transgenic plants comprising a transgene expressing enhanced levels of MKS1 may, by virtue of their increased disease resistance, produce a crop with a larger yield. Furthermore, the crop yield of these transgenic plants will be less dependent on the application of fungicides and bactericides, which are expensive and often have a negative environmental impact. The SAR response is common to many members of the plant kingdom and hence the use of MKS1 proteins to up-regulate the pathogen defence response in a wide range of plants lies within the scope of the present invention.

1. A transgenic plant with enhanced disease resistance and increased expression of a positive regulator of systemic acquired resistance (SAR) characterised by a transgene encoding a MAP kinase substrate 1 (MKS1) polypeptide having a primary amino acid sequence comprising:
a. MAP kinase interaction domain 1 with sequence:
IXGPRPXPLXVXXDSHXIKK, and
b. transcription factor interaction domain 2 with sequence:
PVVIYXXSPKVVHXXXXEFMXVVQRLTG, or
conservatively modified variants of said domain 1 and/or domain 2 sequence, wherein X refers to any amino acid residue.

2. The transgenic plant of claim 1, wherein said MKS1 polypeptide has an amino acid sequence selected from the group consisting of: SEQ ID No. 2, 6, 10, 14, 16, 20, 26, 27, 28, and conservatively modified variants thereof.

3. The transgenic plant of claim 2, wherein said MKS1 polypeptide is encoded by a nucleic acid molecule having a nucleic acid sequence selected from the group consisting of: SEQ ID No. 1, 5, 9, 13, 15, and 19.

4. A method for producing a transgenic plant having enhanced disease resistance, comprising: introducing into the genome of a plant a nucleic acid molecule that hybridizes at high stringency to a nucleic acid molecule having a nucleic acid sequence selected from the group consisting of: SEQ ID No. 1, 5, 9, 13, 15, and 19, as a transgene to produce a transgenic plant, wherein said plant has enhanced disease resistance and increased expression of a positive regulator of systemic acquired resistance.

5. The method of claim 4, wherein said nucleic acid sequence is selected from the group consisting of: SEQ ID No. 1, 5, 9, 13, 15, and 19.

6. The transgenic plant of claim 1, wherein said transgene comprises a homologous promoter.

7. The transgenic plant of claim 1, wherein said transgene is a chimeric gene comprising a heterologous promoter.

8. The transgenic plant of claim 7, wherein said heterologous promoter is selected from the group consisting of: constitutive promoter, tissue specific promoter, and inducible promoter.

9. The transgenic plant of claim 1, wherein said plant is a dicotyledonous plant.

10. The transgenic plant of claim 1, wherein said plant is a monocotyledonous plant.

11. The transgenic plant of claim 1, wherein the plant is selected from the group consisting of: alfalfa, carrot, cotton, potato, sweet potato, oilseed rape, radish, soybean, sugarbeet, sugar cane, sunflower, tobacco, turnip, asparagus, bean, carrot, chicory coffee, celery, cucumber, eggplant, fennel, leek, lettuce, garlic, onion, papaya, pea, pepper, spinach, squash, pumpkin, tomato, brussel sprout, broccoli, cabbage, cauliflower, avocado, banana, blackberry, blueberry, grape, mango, melon, nectarine, orange, papaya, pineapple, raspberry, strawberry, apple, apricot, peach, pear, cherry, plum and quince; herbs such as anise, basil, bay laurel, caper, caraway, cayenne pepper, celery, chervil, chives, coriander, dill, horseradish, lemon balm, liquorice, marjoram, mint, oregano, parsley, rosemary, sesame, tarragon and thyme, eucalyptus, oak, pines and poplar.

12. The transgenic plant of claim 10, wherein the plant is selected from the group consisting of: barley, maize, oats, rice, rye, sorghum, wheat, and Poaceae grass.

13. The transgenic plant of claim 12, wherein said plant is a Poaceae grass selected from the group consisting of Phleum spp., Dactylis spp., Lolium spp., Festulolium spp., Festuca spp., Poa spp., Bromus spp., Agrostis spp., Arrhenatherum spp., Phalaris spp., and Trisetum spp., for example, Phleum pratense, Phleum bertolonii, Dactylis glomerata, Lolium perenne, Lolium multiflorum, Lolium multiflorum westervoldicum, Festulolium braunii, Festulolium loliaceum, Festulolium holmbergii, Festulolium pabulare, Festuca pratensis, Festuca rubra, Festuca rubra rubra, Festuca rubra commutata, Festuca rubra trichophylla, Festuca duriuscula, Festuca ovina, Festuca arundinacea, Poa trivialis, Poa pratensis, Poa palustris, Bromus catharticus, Bromus sitchensis, Bromus inermis, Deschampsia caespitosa, Agrostis capilaris, Agrostis stolonifera, Arrhenatherum elatius, Phalaris arundinacea, and Trisetum flavescens.

14. Seed from the transgenic plant of claim 1.

15. A method for producing the transgenic plant of claim 1, comprising: introducing an expression cassette comprising said transgene encoding said MKS1 polypeptide into a plant; and selecting the transgenic plant or its progeny expressing said MKS1 polypeptide.

16. The method of claim 15, wherein the expression cassette is introduced into the plant through transformation.

17. The method of claim 15, wherein the expression cassette is introduced into the plant by sexual crossing with a transformed plant comprising a MKS1 transgene.

18. A recombinant vector comprising the transgene of claim 1.

19. A method for detecting increased expression of MKS1 polypeptide in the transgenic plant of any one of claim 1, comprising: reacting an anti-MKS1 antibody with a protein extract derived from said plant.

20. An anti-MKS1 antibody or fragment thereof, wherein the antibody or fragment is characterised by reacting with a MKS1 polypeptide having an amino acid sequence selected from the group consisting of: SEQ ID No. 2, 6, 10, 14, 16, 20, 26, 27, 28, and conservatively modified variants thereof.

21. The anti-MKS1 antibody or fragment of claim 20, wherein the antibody is a polyclonal antibody or fragment thereof.

22. The anti-MKS1 antibody or fragment of claim 20, wherein the antibody is a monoclonal antibody or fragment thereof.

23. A method for producing a disease-resistant crop, comprising: cultivating a crop of transgenic plants of claim 1; and harvesting the crop of disease resistant plants.

24. A crop produced by the method of claim 23.

25. A method of breeding a disease resistant plant, comprising:
a) crossing a transgenic plant of claim 1 with a second plant; and
b) screening plants produced by the crossing for retention of the transgene of claim 1; and
c) repeating steps a) and b) to produce a plant having the disease resistance of the plant of claim 1 and at least one characteristic of the second plant.

26. A plant selected in the breeding method of claim 25, wherein said transgene encodes an amino acid sequence selected from the group consisting of: SEQ ID No. 2, 6, 10, 14, 16, 20, 26, 27, 28, and conservatively modified variants thereof.


SEQUENCE LISTING (28 sequences)

SEQ ID NO: 1 - DNA, 669 bp, Arabidopsis sp.
atggatccgtcggagtattttgccggcggcaatccttccgatcaacagaaccagaagcggcagcttcagatctgtggtcctcgtccttcacctcttagtgttcacaaagactctcacaaaatcaagaaacctccaaaacaccctgcgccgccgccaaatcgtgaccaaccgccgccgtatattcctagagagccggtggttatctacgccgtatcccccaaggttgtacacgcaaccgcgtctgagttcatgaacgtagtccagcgactcacagggatctcctctggtgttttcctcgaatctggcggcggtggagatgtttcaccggcggcgaggctagcgtccacggaaaatgctagtccaagaggaggaaaagaaccggctgcgagagatgagacggtggaaatcaacacggctatggaagaagcagctgaatttggtggttatgctccgggaatactctcgccatctccggccttgttgccaacagcttctaccgggatattctctccgatgtatcatcaaggtgggatgttttcgccggctataccactgggattattctcgccggcgggatttatgagcccgtttcgaagtcctggctttactagtttggtagcttcaccaacttttgctgatttctttagtcatatttgggatcaagattag

SEQ ID NO: 2 - protein, 222 aa, Arabidopsis sp.
MDPSEYFAGGNPSDQQNQKRQLQICGPRPSPLSVHKDSHKIKKPPKHPAPPPNRDQPPPYIPREPVVIYAVSPKVVHATASEFMNVVQRLTGISSGVFLESGGGGDVSPAARLASTENASPRGGKEPAARDETVEINTAMEEAAEFGGYAPGILSPSPALLPTASTGIFSPMYHQGGMFSPAIPLGLFSPAGFMSPFRSPGFTSLVASPTFADFFSHIWDQD

SEQ ID NO: 3 - DNA, 16 bp, Arabidopsis sp.
atggatccgtcggagt

SEQ ID NO: 4 - DNA, 16 bp, Arabidopsis sp.
ctaatcttcatcccaa

SEQ ID NO: 5 - DNA, 720 bp, Arabidopsis sp.
atggataatagatcgccaagatcaagaggaatcttgggtccgagaccaataccattgaaagtccgtggagattcgcacaagatcatcaagaagccaccactagcgccgccacacccgcaaccacaaccaccacaaacccatcagcaagaaccgtcacaatcgcggccgccacctggtcccgtgattatatacacagtatctcccaggattatccatacacaccctaataacttcatgacattggtccaacgtctcacaggtaaaacctccacctccacaacatcctcctcctattcttcatctacgtcagcaccaaaagacgcgtcaacaatggttgatacatctcatgggttgatatctccggcggctcggtttgctgttacagagaaggctaatatctcaaacgaactagggacatttgttggaggcgaagggactatggatcaatattatcattatcatcatcatcatcatcatcaagaacaacaacatcaaaatcaagggttcgagcggccaagtttccaccatgctgggattttatcgccgggacctaattctctgccgtcggtatcaccggacttcttttccactattggaccaaccgatccacaaggtttttcgtcgttctttaatgactttaactctatccttcagagtagtccatcgaagattcagtctccttcttctatggaccttttcaacaatttctttgattcttga

SEQ ID NO: 6 - protein, 239 aa, Arabidopsis sp.
MDNRSPRSRGILGPRPIPLKVRGDSHKIIKKPPLAPPHPQPQPPQTHQQEPSQSRPPPGPVIIYTVSPRIIHTHPNNFMTLVQRLTGKTSTSTTSSSYSSSTSAPKDASTMVDTSHGLISPAARFAVTEKANISNELGTFVGGEGTMDQYYHYHHHHHHQEQQHQNQGFERPSFHHAGILSPGPNSLPSVSPDFFSTIGPTDPQGFSSFFNDFNSILQSSPSKIQSPSSMDLFNNFFDS

SEQ ID NO: 7 - DNA, 20 bp, Arabidopsis sp.
atggataatagatcgccaag

SEQ ID NO: 8 - DNA, 21 bp, Arabidopsis sp.
tcaagaatcaaagaaattgtt

SEQ ID NO: 9 - DNA, 791 bp, Brassica oleracea
taatttttcccttttttttttgtttataaatgttttggtcaatactagctcgtcgtcgacaaagattcatttcgattcccaaaccacacaagaagaacacaaattagctcgaaagaaacaaactcttttgagaaaataatggatccgtcggagtctttcgccggcggcaatccttccgaccaacagaaccagaaacgtcagcttcagatctgtggtcctcgtccctcacctctcagcgtcaacaaagactctcacaagatcaagaaacctcctaaacaccctgctcctccgcctcagcatcgcgaccaagctccgctctacgctgctcgagagccggtggtcatctacgccgtctcgccgaaagtcgtccacaccacagcctcggatttcatgaacgtcgtccagcgtctcaccggcatctcatccgccgtcttcctcgaatccggtaacggcggagatgtatctccggcggcgagactcgccgcgaccgagaatgcaagcccgagaggaggaaaagaaccggtgatggcggctaaagatgagacggtggaaatcgcgacggctatggaagaagcagccgagttgagcggctatgcgccggggatactctccccttctccggctatgttaccgacagcttctgccggaatattctcgcagatgactactcaccaaggtgggatgttctcgccgggattgttttcgccggcggggttaatgagcccgtttggttttgctagcttggttgcttctccaacgtttgctgatttgttcagtcatatttggggata

SEQ ID NO: 10 - protein, 217 aa, Brassica oleracea
MDPSESFAGGNPSDQQNQKRQLQICGPRPSPLSVNKDSHKIKKPPKHPAPPPQHRDQAPLYAAREPVVIYAVSPKVVHTTASDFMNVVQRLTGISSAVFLESGNGGDVSPAARLAATENASPRGGKEPVMAAKDETVEIATAMEEAAELSGYAPGILSPSPAMLPTASAGIFSQMTTHQGGMFSPGLFSPAGLMSPFGFASLVASPTFADLFSHIWG

SEQ ID NO: 11 - DNA, 20 bp, Brassica oleracea
atggatccgtcggagtcttt

SEQ ID NO: 12 - DNA, 20 bp, Brassica oleracea
tatccccaaatatgactgaa

SEQ ID NO: 13 - DNA, 878 bp, Brassica oleracea
aaaagtcaacattttgaaagtcaaactaatcggtctcagaaaacaaaaataactttgtgtgttgatgtttaggtcaatatactcgtcgtcaaaacatcccttcaatttctcagaccaaacacagagaagaaacaagttggatccaaactctctacaacaaaaagtagtgaacgagagaagctctccccaagcgtttaatggatccgtcggagcacttcgccggcggtaatcctttcgatcaacagactccaaaacgtcagcttcagatctgtggccctcgtccttcacctctaagcgtcaacaaagactctcacaagatcaagaaacctcccaggcaccctgctccacctcctcagcatcaccgcgaccaagctccgctctaccctcctcgagagccggtggttatctacgccgtctcgccgaaagtcgtgcacaccacaacctccgatttcatgaacgtcgtccagcgtctcaccgggatctcctccgaggtcttcctcgaatcaagaaacgacggagatgtatcaccggcggcgagactcgccgcgacggagaatgctagcccgagaggaggaaaggaaccggtggaaagctcgacggctatggaagaagcagctgagttcggttgttatgtgccgggaatactctcgccgtctccggctatgttaccgaccgttcccgccggaattttctctccgatgtttcacctaggtgggttgttttcgccggcgttgccgccgggattattttcgccggcaggattaatgagccctggttatgctagtttggcgtcaccaaattttgctgatttcttcagtcacatttgggatccttagagaatagattattagttttttttattatttacattttatgta

SEQ ID NO: 14 - protein, 212 aa, Brassica oleracea
MDPSEHFAGGNPFDQQTPKRQLQICGPRPSPLSVNKDSHKIKKPPRHPAPPPQHHRDQAPLYPPREPVVIYAVSPKVVHTTTSDFMNVVQRLTGISSEVFLESRNDGDVSPAARLAATENASPRGGKEPVESSTAMEEAAEFGCYVPGILSPSPAMLPTVPAGIFSPMFHLGGLFSPALPPGLFSPAGLMSPGYASLASPNFADFFSHIWDP

SEQ ID NO: 15 - DNA, 393 bp, Glycine max
caacttcaaggtccacgccctacacctctcagaataaacaaagactctcataaaatcaagaaaccaccgttggcaccacaaccttcacaccctcatcaacctccaccgcgccaacctataataatctacaccgtgtcccccaaggtgattcacaccaccccaagtgacttcatgaacctcgtccaacgcctcactgggtccagttcttcttcctctgctgaagtggtcatgtccaacaataacaacaccactcatgtcgaccctttcaacaacggcggcggcggaatggtgtcgccggcggcgcgttacgccaccatagagaaggccatgtcccctatggggaaaaaacatgttcttcttccaagtgtgaacaatattataagcgatgtggaa

SEQ ID NO: 16 - protein, 131 aa, Glycine max
QLQGPRPTPLRINKDSHKIKKPPLAPQPSHPHQPPPRQPIIIYTVSPKVIHTTPSDFMNLVQRLTGSSSSSSAEVVMSNNNNTTHVDPFNNGGGGMVSPAARYATIEKAMSPMGKKHVLLPSVNNIISDVE

SEQ ID NO: 17 - DNA, 19 bp, Glycine max
ccatagagaaggccatgtc

SEQ ID NO: 18 - DNA, 20 bp, Glycine max
tgaatgttgtggtgccaacg

SEQ ID NO: 19 - DNA, 927 bp, Oryza sp.
gtggcgatggaattcccgtcgtcgacgtcgccgtcgccgtcgccgtcgtccgggcagcatcagcagcagccgacgacgccgcggcggcagcttcagggcccgcgccccccgcggctcaacgtgcggatggagtcgcacgccatcaagaagccgtcgtccggggcggccgcggcggcggcggcggcgcaggcgaggcgggagcagcagcagccgccgccgcgggcgccggtgatcatctacgacgcgtcgccgaagattatccacgccaagcccaacgagttcatggcgctcgtgcagcggctcaccggcccggggtcggggccgccggcgccgccgcatcaaggggaggcccaggcgcaggactacccgatgatggacgaggccgccgcgcagcagttcttcccgccggagctgctgctctcgccgtcggccgcgatgtccccggcggcgaggctggcgaccatcgagaggtccgtccgcccgatgcccgagccggcgccggagtacgtggacatcacgaacggcggcggcggcggcggggtcgacgacggcggcctcgcggcgatcctcggctcgatccggccaggcatcctctccccgctcccctcctccctcccgcccgccgccgtccccggccagttctcgccgctcccgttcgacgcgaggccgctcccgttcgacgcgagctgcatcagctggctcaacgagctgagccccatcctccgggccgcctccgccggcgcggcctcgtccggcagcggcggcggcggcagcggtggcaacaccagcaacggcggcggcgcccgcccgccgccgtcctactacgccgacccattcgtccccagcccacgtcacctcctcgccacgcccaccgtgccgtcgccggcgacctgcgccgagctcttcagcaacctgccggatctctag

SEQ ID NO: 20 - protein, 306 aa, Oryza sp.
MEFPSSTSPSPSPSSGQHQQQPTTPRRQLQGPRPPRLNVRMESHAIKKPSSGAAAAAAAAQARREQQQPPPRAPVIIYDASPKIIHAKPNEFMALVQRLTGPGSGPPAPPHQGEAQAQDYPMMDEAAAQQFFPPELLLSPSAAMSPAARLATIERSVRPMPEPAPEYVDITNGGGGGGVDDGGLAAILGSIRPGILSPLPSSLPPAAVPGQFSPLPFDARPLPFDASCISWLNELSPILRAASAGAASSGSGGGGSGGNTSNGGGARPPPSYYADPFVPSPRHLLATPTVPSPATCAELFSNLPDL

SEQ ID NO: 21 - DNA, 16 bp, Oryza sp.
atggaattcccgtcgt

SEQ ID NO: 22 - DNA, 19 bp, Oryza sp.
ctagagatccggcaggttg

SEQ ID NO: 23 - DNA, 781 bp, CaMV 35S promoter duplicated
atggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctct

SEQ ID NO: 24 - DNA, 253 bp, Agrobacterium NOS terminator
cgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggg

SEQ ID NO: 25 - DNA, 189 bp, Synthetic intron
gtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcag

SEQ ID NO: 26 - protein, 207 aa, Oryza sp.
MEQQLSSPSASQRGGGRELQGPRPAPLKVRKESHKIRKQEPVQQLRQPVIIYTMSPKVVHANAADFMSVVQRLTGAPPTAPPQPQPHHPTLLAQMPPQPSFPFHLQQQDAWPQQQHSPAAIEQAAARSSGADLPPLPSILSPVPGTVLPAIPASFFSPPSLISPVPFLGATTTSSAAPSTSPSPMGGSAYYWDLFNMQQQQHYHHQN

SEQ ID NO: 27 - protein, 238 aa, Zea mays
MDPPSSSGRPTTPRRQLQGPRPPRLNVRMESHAIKKPSASGAPPAPGQGRPRDHHHHHPQPGRAPVIIYDASPKVIHAKPSEFMALVQRLTGPGAQAQHERHVADDDATANGGGVLGQAFLPPELLLSPSAAMSPAARLATIERSVRPVPAPAPAPDYAADGHPRGGARPREAPRHPVPAAVLAAAGRRVGPVLAAALRPQQRQLAQRAQPHPPGSVHGQRSAPLAHAHGPTGGSRQP

SEQ ID NO: 28 - protein, 271 aa, Zea mays
QGPRPPRLAVSKDSHKVRKPPVAPQRQQHQHQQPAAQLQQQQHQYHQQQQQQGRQPVIIYDASPKVIHTKPGDFMALVQRLTGPGSTSQAQFDAAAAAAGPSHPAAMEFEPREFLLSPTAALSPAARLAAIERSVRPLPPHHAPAAVPPYFGATNDDGFFLPGSADMDSLSAALGPPAGRPGILSPAALPPAASTGLFSPMPFDPSCLSWLSELSPFLPSAGTRAAAAGLLDQAPFAPSPRSSLLLSTPTMPSPATFSVLEFFSSPNFPDL